(19) United States
(12) Patent Application Publication
(10) Pub. No.: US 2012/0029197 A1
(43) Pub. Date: Feb. 2, 2012

(54) Method for Preparing Sulfur-Containing Compounds

(75) Inventors: Adamo Mauro, Dublin (IE); Nagabelli Murali, Andhrapradesh (IN)

(73) Assignee: THE ROYAL COLLEGE OF SURGEONS IN IRELAND, Dublin (IE)

(21) Appl. No.: 13/139,475

(22) PCT Filed: Dec. 11, 2009
(86) PCT No.: PCT/EP2009/008890

§ 371 Date: Oct. 6, 2011

(30) Foreign Application Priority Data
Dec. 11, 2008 (EP) 08021530.4
Jun. 22, 2009 (GB) 0910726.9

(51) Int. Cl. C07D 453/00

(52) U.S. Cl. 546/134

(57) ABSTRACT

The invention provides a method for preparing sulfur-containing compounds, the method comprising reacting a donor compound comprising at least one sulfur having at least one lone pair of electrons, with an acceptor compound; wherein the reaction occurs in the presence of an amine, optionally an amine catalyst, capable of activating the sulfur having at least one lone pair of electrons; and wherein the reaction occurs via the formation of an transient intermediate species, optionally a transient intermediate species, between the amine, optionally the amine catalyst and the donor compound; and wherein the donor compound is selected from the group consisting of a sulfurous acid, a sulfenic acid and a sulfinic acid or a salt, ester or amide of a sulfurous acid, a sulfenic acid and a sulfinic acid. The invention also provides sulfur-containing compounds of the formula: wherein R is selected from: (a) 1-(4-Nitro-phenyl)-3-oxo-3-phenyl-propane; (b) 2-(3-Methyl-4-nitro-isoxazol-5-yl)-1-phenyl-ethane; (c) 1-(4-Methoxy-phenyl)-2-(3-methyl-4-nitro-isoxazol-5-yl)-ethane; (d) 2-(3-Methyl-4-nitro-isoxazol-5-yl)-1-(4-nitro-phenyl)-ethane; (e) 1-(4-Fluoro-phenyl)-2-(3-methyl-4-nitro-isoxazol-5-yl)-ethane; (f) 1-(4-Chloro-phenyl)-2-(3-methyl-4-nitro-isoxazol-5-yl)-ethane; and (g) 3-Oxo-cyclohexane. Finally, the invention provides use of chiral sulfur-containing compounds obtainable by the above-mentioned method or chiral sulfur-containing compounds as mentioned above for the resolution of racemic mixtures of amines.

This invention relates to methods for preparing sulfur-containing compounds, such as sulfonic acid compounds. Optionally, it relates to methods for preparing heterochiral sulfur-containing compounds, such as heterochiral sulfonic acid compounds, as well as sulfones and sulfoxides, sulfones arising from the addition of sulfinic acids to alkenes and sulfoxides arising from the addition of sulfenic acids to alkenes and alkynes.

### BACKGROUND TO THE INVENTION

Reaction of bisulfite with α,β-unsaturated ketones, esters, and amides has been known for over a century (Scheme 1) [(a) Beilstein, F. K. et Al. Chem. Ber. 1885, 18, 482. (b) Dodge, F. D. J. Am. Chem. Soc. 1930, 52, 1724].

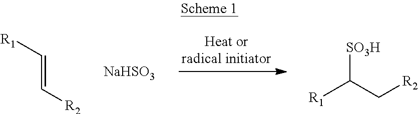

However, there are few reported uses of this reaction. Most examples used highly activated disubstituted enones, require long reaction times, or the use of high temperatures and microwave. Alternatively, a radical initiator is employed in combination with high temperature. Taken together, it is evident that the addition of bisulfite to α,β-unsaturated ketones, esters, and amides occurs only under enforcing conditions, and therefore is unpractical. Additionally, the stringency of the required conditions imposes limits on the range of α,β-unsaturated ketones, esters, and amide substrates for use in the reaction.

Sulfonic acids, sulfones and sulfoxides are present in a wide number of marketed compounds. For example, taurine is present is several soft drinks. Presently, the production of sulfonic acids involves the reaction of alkenes and bisulfite at high temperature and/or in the presence of radical initiators. The use of flammable compounds (alkenes) in gas phases at high temperature and/or the use of radical species raise both the risk of explosions and the manufacturing cost. It would be advantageous to prepare sulfonic acids, sulfones and sulfoxides without the need for heating-cooling equipment and without the need for radical chemistry, making the preparation of the sulfur-containing compounds safer, cleaner and more cost efficient.

At present, no enantioselective version of this reaction has been reported. Chiral sulfonic acids are also employed for the resolution of racemic mixtures of amines. When the chiral pool cannot be employed, resolution is the most frequently alternative used by industry to obtain single enantiomers. “Dutch resolution” is a technique for the resolution of racemates that makes use of families of resolving agents. However, there exists only a few families of resolving agents.

Commonly used compounds such as quinine, brucine, camphor sulfonic acid, and bromocamphor sulfonic acid tend to be rare. This means that, at present, two steps are required (addition of bisulfite to alkenes and subsequent resolution) to obtain chiral sulfonic acids.

Racemic sulfonic acids, also known as alpha olefin sulfonates (AOS), are a type of anionic surfactant capable of excellent emulsifying, decontaminating, and calcium soap dispersing performances. Advantages include good solvency and compatibility, rich and fine foam, easy biodegradation, low toxicity and low irritation to skin. Especially in the application of non-phosphorus detergents, sulfonic acids have not only the good washing ability, but also good compatibility with enzyme agents. Powder (grain) shape products have good fluidity, therefore they are widely used in non-phosphorus washing powder, liquid detergents and home washing products, textile, printing and dyeing industry, petrochemical products, and industrial hard surface cleaning agents.

### SUMMARY OF THE INVENTION

According to a first aspect of the present invention, there is provided a method for preparing sulfur-containing compounds, the method comprising reacting

a donor compound comprising at least one sulfur having at least one lone pair of electrons,

with an acceptor compound;

wherein the reaction occurs in the presence of an amine, optionally an amine catalyst, capable of activating the sulfur having at least one lone pair of electrons; and wherein the reaction occurs via the formation of an intermediate species, optionally a transient intermediate species, between the amine, optionally the amine catalyst, and the donor compound; and wherein the donor compound is selected from the group consisting of a sulfurous acid, a sulfenic acid and a sulfinic acid, or a salt, ester or amide of a sulfurous acid, a sulfenic acid and a sulfinic acid.

By “intermediate species” is meant true compounds in which the amine and the donor compound are covalently linked to form an intermediate compound that be transient in its nature, as well as species in which the amine and the donor compound are not covalently linked but are more loosely associated to an intermediate species that be transient in its nature.

The reaction may be carried out using any suitable procedure, wherein the donor compound, the acceptor compound, and the catalyst are reacted under suitable conditions to provide the required sulfur-containing compounds.

By the term “sulfur-containing compounds” is meant an acid comprising at least one sulfur atom, and esters thereof or amides thereof, as well as sulfones and sulfoxides. Optionally, the sulfur-containing compound is an organic compound. Non-limiting examples of sulfur-containing compounds include sulfonic acids, sulfinic acids, sulfenic acids, and esters thereof or amides thereof.

Advantageously, the reaction occurs without the requirement of providing a radical initiator. By “radical initiator” is meant a substance capable of producing a radical species, preferably under mild reaction conditions. More specifically, the reaction occurs in the presence of less than 0.001% (g/g) radical initiator. Typical radical initiators include hydrogen peroxide, benzoyl peroxide, tert-butylperoxide, samarium iodide, cerium ammonium nitrate and their combination with light and heating. Preferably, the reaction occurs in the presence of no radical initiator. The absence of the need for radical initiators to be involved in the methods of the present invention is supported by the reaction proceeding in the presence of radical scavengers and at room temperature, as is exemplified hereunder.

Optionally or additionally, the reaction occurs in the presence of a reaction solvent. Optionally, the reaction solvent is inert.

Optionally, the reaction solvent is aprotic. It will be appreciated that aprotic solvents cannot donate hydrogen. Polar aprotic solvents are solvents that share ion-dissolving power with protic solvents but lack an acidic hydrogen. Polar aprotic solvents generally have high dielectric constants and high polarity. Examples are dimethyl sulfoxide, toluene, dimethylformamide, dioxane and hexamethylphosphorotriamide and tetrahydrofuran. Further optionally, the reaction solvent is tetrahydrofuran.

Alternatively, the reaction solvent is protic, such as methanol and water, or a mixture thereof, of which methanol is preferred.

Optionally, the at least one sulfur having at least one lone pair of electrons is capable of being transferred to the acceptor compound, via the formation of an intermediate species, optionally a transient intermediate species, such as an intermediate compound, optionally a transient intermediate compound.

Further optionally, the reaction could be carried out in a biphasic system, in which, for example, the donor compound is in one phase and the acceptor compound is in another phase. One such biphasic system is water/toluene and one such biphasic catalyst might be an ammonium salt, for example Bu₄NOH. For example, the donor compound might be in the aqueous phase and the acceptor compound in the toluene phase.

Donor Compound

The term “a donor compound comprising at least one sulfur having at least one lone pair of electrons” is intended to include bisulfite, sulfinic and sulfenic acids. For example, the donor compound is selected from a sulfurous acid, a sulfenic acid and a sulfinic acid or a salt, ester or amide of a sulfurous acid, a sulfenic acid and a sulfinic acid. Those skilled in the art will appreciate that a salt of a sulfurous acid is a bisulfite. R groups, when present, could be any alkyl or aryl or heteroaryl group:

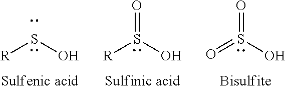

Optionally, the at least one sulfur having at least one lone pair of electrons is capable of being transferred to the acceptor compound. The sulfur having at least one lone pair of electrons can be transferred via the formation of an intermediate species or compound, for example an intermediate compound.

Preferably, the at least one sulfur having at least one lone pair of electrons is selected from a sulfite anion, a sulfonate anion or a sulfenate anion, for example, a sulfite anion.

Preferably, the donor compound is selected from sodium hydrogen sulfite (sodium bisulfite), phenylsulfinic acid and antraquinone sulfenic acid, for example, sodium hydrogen sulfite (sodium bisulfite).

Acceptor Compound

By the term “acceptor compound” is meant an unsaturated compound or a cyclic derivative thereof. For example, a cyclic derivative of an alkene comprises an epoxide or an aziridine.

By the term “unsaturated compound” is meant a polyatomic compound, wherein the bond order between at least one pair of atoms is greater than 1. For the purposes of this specification, an unsaturated compound is intended to include any compound having one more, for example, a double-, triple-, or higher order-bond between at least one pair of atoms. The term “multiple bond” is intended to include any double-, triple-, or higher order-, bond. Non-limiting examples include an alkene or an alkyne. The at least one multiple bond extends between two adjacent atoms that may be the same atom or different atoms. Preferably, at least one atom is a carbon atom. Further preferably, both atoms are carbon atoms. Alternatively, at least one atom in the at least one multiple bond is a heteroatom such as, nut not limited to, nitrogen, oxygen and sulphur.

For the purposes of this specification, in the case of a polyatomic compound represented by text, a single bond extending between any two atoms is represented by a solid dashed line (—), a double bond extending between any two atoms is represented by a double solid dashed line (═), and a triple bond extending between any two atoms is represented by a triple solid dashed line (≡), unless otherwise stated.

Optionally or additionally, the unsaturated compound comprises at least one, such as a, functional group selected from the group comprising, but not limited to, (C═NH), (C═N—R), (C═NR₁R₂), (C═O), (C═O), (C═S), and (C═S).

Further optionally, the unsaturated compound is selected from the group comprising, but not limited to, an imine (comprising the functional group C═NH), an N-substituted imine (comprising the functional group C═N—R), an iminium ion (comprising the functional group C═NR₁R₂), an aldehyde (comprising the functional group C═O), a ketone (comprising the functional group C═O), a thioaldehyde (comprising the functional group C═S), and a thioketone (comprising the functional group C═S).

When the acceptor compound is an unsaturated compound, at least one electron-withdrawing group is adjacent the at least one multiple bond. By “electron-withdrawing group” is meant any group that causes uneven electron distribution within the at least one multiple bond. Such “electron-withdrawing groups” include, but are not limited to, carbonyl groups, and their esters; nitro groups; cyano groups; oximes; hydrazones; imino groups; protected imino groups such as BOC or CBZ protected imines.

Where the acceptor compound is a cyclic derivative of an unsaturated compound, it is not thought necessary to have at least one electron-withdrawing group adjacent the cyclic derivative. Without being bound by theory, it is thought that the electrons within the cyclic derivative are already unevenly distributed, making the presence of an adjacent electron-withdrawing group unnecessary.

Optionally, the unsaturated compound may be linear or branched. Preferably, the unsaturated compound is an alkene or an alkyne, for example, an alkene. Alternatively, the unsaturated compound is an arene.

Alternatively, the unsaturated compound may be cyclic. The unsaturated compound may be monocyclic, polycyclic, or heterocyclic. Polycyclic unsaturated compounds may also include compounds having fused rings.

Optionally, the unsaturated compound is selected from the group comprising, but not limited to, molecules 1a (1,3-diphenyl-propenone); 1b (3-(4-methoxy-phenyl)-1-phenyl-propenone); 1c (3-(4-nitro-phenyl)-1-phenyl-propenone); 3a (3-methyl-4-nitro-5-styryl-isoxazole); 3b (5-[2-(4-methoxy-phenyl)-vinyl]-3-methyl-4-nitro-isoxazole); 3c (3-methyl-4-nitro-5-[2-(4-nitro-phenyl)-vinyl]-isoxazole); 3d (5-[2-(4-fluoro-phenyl)-vinyl]-3-methyl-4-nitro-isoxazole); 3e (5-[2-(4-chloro-phenyl-vinyl]-3-methyl-4-nitro-isoxazole); 5 ((2-nitro-vinyl)-benzene); 7 (but-3-en-2-one); 9 (4-methyl-pent-3-en-2-one); 11 (3-phenyl-acrylic acid ethyl ester); 13 ([1,2]naphthoquinone); 15 ([1,4]naphthoquinone); 17 (cyclohex-2-enone); and); and 23 (3-phenyl-acrylonitrile) of Table 2.

Further optionally, the unsaturated compound is selected from the group comprising, but not limited to, molecules 48 (benzylidene-phenyl-amine), 50a (benzaldehyde), 50b (1-Phenyl-ethanone), and 52 (antraquinone sulfenic acid) of Examples 7, 8, and 10 respectively.

By the term “cyclic derivatives of alkenes” is meant an epoxide or an aziridine. Optionally, the epoxide is linear or branched. Optionally, the epoxide is monosubstituted, disubstituted, trisubstituted or tetrasubstituted. One suitable epoxide is styrene oxide. Optionally, the aziridine is monosubstituted, disubstituted, trisubstituted or tetrasubstituted. Optionally, the nitrogen of the aziridine is substituted with one or more acyl, aryl or alkyl groups. Preferably, the nitrogen of the aziridine is a non-substituted (H).

Without being bound by theory, it is thought that the likely reaction mechanism for epoxides or aziridines as acceptor compounds is set forth below:

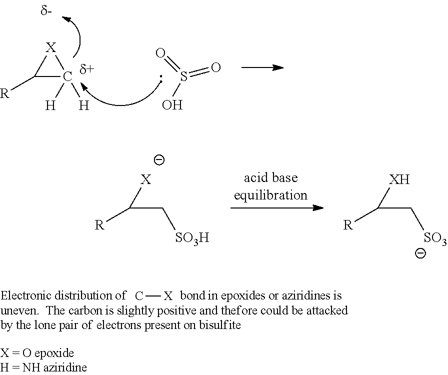

Catalyst

The catalyst capable of activating the sulfur having at least one lone pair of electrons acts both to deprotonate the donor compound having at least one sulfur having at least one lone pair of electrons, so that the donor compound then becomes nucleophilic enough to attack the acceptor compound and, additionally, to impart an acceleration to the reaction. The acceleration is carried out by forming a salt with the donor compound, as will be exemplified hereunder. In this context, formation of specific hydrogen bond activates the donor compound to nucleophilic addition. It is thought that the proton in bisulfite, for example, is located on the sulfur (structure B below). However, structure B is in equilibrium with structure A (also below), which is the reactive species in the sulfonylation reaction observed. The catalyst (amine in certain embodiments) is able to stabilise the most reactive species A that is present in increased amount. The enhanced concentration of species A leads to an enhanced reaction rate, producing a net activation of bisulfite.

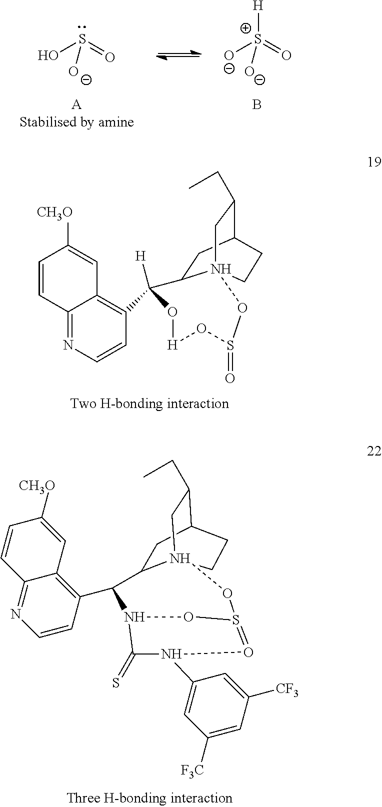

It also thought that a sulfonate anion or a sulfenate anion would be similarly activated.

By the term “activating” is meant stabilising the sulfonylation reactive species (such as a sulfite anion, a sulfonate anion or a sulfenate anion), possibly by hydrogen bond interactions with the catalyst (amine in certain embodiments).

Optionally, the catalyst is a heterochiral compound, further optionally a homochiral compound. By the term “heterochiral” is meant a chiral compound having an enantiomeric species wherein one enantiomeric form (R or S) is in excess of the other. By the term “homochiral” is meant an enantiomeric species comprising more than 70%, optionally more than 90%, further optionally more than 95%, still further optionally more than 99%, of one enantiomeric form (R or S).

Preferably, the catalyst is a homochiral compound.

Preferably, the catalyst is a nucleophile.

Optionally, the nucleophile is a heterochiral compound, further optionally a homochiral compound. Preferably, the nucleophile is a homochiral compound.

Optionally, the nucleophile comprises at least one valence electron that does not form part of a covalent bond. Preferably, the nucleophile comprises at least one pair of valence electrons, wherein the at least one pair of electrons does not form part of a covalent bond.

Preferably, the nucleophile comprises one or more hydrogen bond acceptors, and at least one hydrogen bond donor. Optionally, the nucleophile comprises one or two hydrogen bond acceptors, and one or two hydrogen bond donors. By the term “hydrogen bonds” is meant electrostatic attractions between a hydrogen bearing a partial positive charge and another atom (usually O or N) bearing a partial negative charge. These partial opposite charges are a consequence of the relative electronegativity of covalently-bonded atoms. By the term “hydrogen bond donor” is meant a molecule containing a hydrogen bound to an electronegative element (N, O, S, for example). By the term “hydrogen bond acceptor” is meant a molecule containing atoms having localised non-bonding electron pairs (lone pair).

Preferably, the nucleophile is an amine or, optionally, a salt therof.

Optionally, the amine is a heterochiral compound, further optionally a homochiral compound. Preferably, the amine is a homochiral compound.

Optionally, the nucleophile is a quaternary ammonium compound. The term “quaternary ammonium compound” is synonymous with the terms “quaternary ammonium salt”, and “quaternary amine”, and is intended to include salts formed from a polyatomic compound having a non-neutral charge, also referred to as “polyatomic ions”. The polyatomic ion is, optionally, a quaternary ammonium cation, wherein the polyatomic ion comprises the structure NR₄⁺, wherein R is an alkyl group, and the non-neutral charge is a positive charge.

Optionally, the quaternary ammonium compound is a heterochiral compound, further optionally a homochiral compound. Preferably, the quaternary ammonium compound is a homochiral compound.

Optionally, the amine is a cyclic amine. Further optionally, the amine is a heterocyclic amine.

Optionally, the amine is pyridine.

Preferably, the amine is an aliphatic amine.

Optionally, the amine is a secondary amine.

Preferably, the amine is a tertiary amine. Optionally, the amine comprises at least one nitrogen atom having at least one pair of valence electrons, wherein the at least one pair of electrons does not form part of a covalent bond; and at least three functional groups. Each functional group may be the same functional group or a different functional group. Preferably, each of the three functional groups is a different functional group. Preferably, the amine is triethylamine.

Without being bound by theory, pyridine is an aromatic amine while triethylamine is not, therefore the lone pair in pyridine is delocalised and is thought to be less available to engage in hydrogen-bonding (acceptor). In triethylamine, the lone pair is present stably on the nitrogen and therefore a more tight pair could be formed with bisulfite.

Optionally, the reaction is carried out at between −90° C. and 35° C., optionally either between −20° C. and 5° C. or between 18° C. and 22° C.

Optionally, the reaction is carried out for a period of time such that an isolatable amount of the sulfur-containing compound is produced. Optionally, the period of time is between 20 min and 96 h, optionally between 1 h and 40 h, further optionally between 1 h and 20 h.

Optionally, 0.01 equiv to 10 equiv of catalyst is provided, per equiv of the donor compound. By the term “equiv” is meant molar equivalents. For example, 0.05 equiv to 10 equiv, optionally 0.05 equiv to 5 equiv, further optionally 0.05 equiv to 1 equiv, still further optionally 0.05 equiv to 0.5 equiv, of catalyst is provided, per equiv of the donor compound.

Optionally, the reaction rate is improved by reducing the concentration of molar equivalents of amine (or amine catalyst) to donor compound. It is thought that the reaction rate is optimised when the amine (or amine catalyst) is present in a molar ratio of amine:donor compound of about 1:20 to about 1:2000, although the reaction will still proceed when the amine (or amine catalyst) is present in a molar ratio of amine:donor compound of about 1:10 to 1:2000 such as, optionally, about 1:10 to about 1:30, for example about 1:22.

In a preferred embodiment of the present invention, there is provided a method for preparing heterochiral, optionally homochiral, sulfur-containing compounds, in which the method comprises reacting

a donor compound comprising at least one sulfur having at least one lone pair of electrons with

an acceptor compound;

wherein the reaction occurs in the presence of a heterochiral, optionally homochiral, amine, optionally an amine catalyst capable of activating the sulfur having at least one lone pair of electrons; and wherein the reaction occurs via the formation of an intermediate species, optionally a transient intermediate species, between the amine, optionally the amine catalyst, and the donor compound; and wherein the donor compound is selected from the group consisting of a sulfurous acid, a sulfenic acid and a sulfinic acid or a salt, ester or amide of a sulfurous acid, a sulfenic acid and a sulfinic acid.

Optionally, the heterochiral, optionally homochiral, catalyst is a heterochiral, optionally homochiral, amine. Further optionally, the heterochiral, optionally homochiral, amine is selected from the group comprising, but not limited to, molecules 19 (5-ethyl-1-aza-bicyclo[2.2.2]oct-2-yl)-(6-methoxy-quinolin-4-yl)-methanol); 19a (1-(3,5-bis-trifluoromethyl-phenyl)-3-[(5-ethyl-1-aza-bicyclo[2.2.2]oct-2-yl)-(6-methoxy-quinolin-4-yl)-methyl]-thiourea); 20 (pyrrolidin-2-yl-methanol); and 21 (2-amino-2-phenyl-ethanol).

Preferably, the heterochiral, optionally homochiral, catalyst is a heterochiral, optionally homochiral, quaternary ammonium compound. Further preferably, the heterochiral, optionally homochiral, quaternary ammonium compound is selected from the group comprising, but not limited to, molecules 31 4-[(5-Ethyl-1-aza-bicyclo[2.2.2]oct-2-yl)-methoxy-methyl]-6-methoxy-quinoline, 32-[(5-Ethyl-1-aza-bicyclo[2.2.2]oct-2-yl)-methoxy-methyl]-6-methoxy-quinoline, 33 Benzoic acid (5-ethyl-1-aza-bicyclo[2.2.2]oct-2-yl)-(6-methoxy-quinolin-4-yl)-methyl ester; 34 Benzoic acid (5-ethyl-1-aza-bicyclo[2.2.2]oct-2-yl)-(6-methoxy-quinolin-4-yl)-methyl ester; 35 Benzoic acid (5-ethyl-1-aza-bicyclo[2.2.2]oct-2-yl)-(6-methoxy-quinolin-4-yl)-methyl ester; 36 Phenyl-carbamic acid (5-ethyl-1-aza-bicyclo[2.2.2]oct-2-yl)-(6-methoxy-quinolin-4-yl)-methyl ester; 37 [(5-Ethyl-1-aza-bicyclo[2.2.2]oct-2-yl)-(6-methoxy-quinolin-4-yl)-methyl]-phenyl-amine; 38 [(5-Ethyl-1-aza-bicyclo[2.2.2]oct-2-yl)-(6-methoxy-quinolin-4-yl)-methyl]-phenyl-amine; 39 1-(3,5-Bis-trifluoromethyl-phenyl)-3-[(5-ethyl-1-aza-bicyclo[2.2.2]oct-2-yl)-(6-methoxy-quinolin-4-yl)-methyl]-thiourea; 40 3-{[(5-Ethyl-1-aza-bicyclo[2.2.2]oct-2-yl)-(6-methoxy-quinolin-4-yl)-methyl]-amino}-4-phenylamino-cyclobut-3-ene-1,2-dione; 41 3-{[(5-Ethyl-1-aza-bicyclo[2.2.2]oct-2-yl)-(6-methoxy-quinolin-4-yl)-methyn-amino}-4-phenylamino-cyclobut-3-ene-1,2-dione; 42 2-[3-(2-Amino-cyclohexyl)-thioureido]-N,N-dimethyl-propionamide; and 43 1-(3,5-Bis-trifluoromethyl-phenyl)-3-(2′-dimethylamino-[1,1′]binaphthalenyl-2-yl)-thiourea of Table 4; or the group comprising, but not limited to, molecules 44 1-Benzyl-5-ethyl-2-[hydroxy-(6-methoxy-quinolin-4-yl)-methyl]-1-ammonium-bicyclo[2.2.2]octane chloride; 45 1-Benzyl-5-ethyl-2-(hydroxy-quinolin-4-yl-methyl)-1-ammonium-bicyclo[2.2.2]octane chloride; 46 1-Benzyl-5-ethyl-2-[hydroxy-(6-methoxy-quinolin-4-yl)-methyl]-1-ammonium-bicyclo[2.2.2]octane; and 47 1-Benzyl-5-ethyl-2-(hydroxy-quinolin-4-yl-methyl)-1-ammonium-bicyclo[2.2.2]octane chloride of Table 5a or 5b.

By the term “heterochiral” is meant a chiral compound having an enantiomeric species wherein one enantiomeric form (R or S) is in excess of the other.

Optionally, the sulfur-containing compounds are substantially homochiral. By substantially homochiral is meant an enantiomeric species comprising more than 70%, optionally more than 90%, further optionally more than 95%, still further optionally more than 99%, of one enantiomeric form (R or S).

Without being bound by theory, it is thought that lowering the reaction temperature to 5° C. or below may increase the enantiomeric excess. Indeed, Example 3 hereunder demonstrates that lowering the reaction temperature to 0-5° C. from room temperature does improve the enantiomeric excess.

In addition, it is thought that the dielectric constant of the reaction solvent may influence the enantiomeric excess obtained. Suitable aprotic reaction solvents include, but are not limited to toluene, tetrahydrofuran (close to 0), hexane (1.9), dioxane (2.2), chloroform (4.8), acetonitrile (37), and dimethyl sulfoxide (47). Suitable protic solvents include, but are not limited to, butanol (12.5), ethanol (24.5), and methanol (32.7), of which methanol is preferred. Also contemplated as suitable reaction solvents, are combinations of aprotic reaction solvents and protic solvents. Optionally, the suitable reaction solvent comprises a combination of at least one aprotic reaction solvent independently selected from toluene, tetrahydrofuran, hexane, dioxane, chloroform, acetonitrile, and dimethyl sulfoxide; and at least one protic solvent independently selected from butanol, ethanol, and methanol.

Without being bound by theory, it is thought that ionic couples form more quickly in polar solvents, as the polar ions have to travel around the media to find each other. However, ionic couples are thought to be more stable in aprotic and apolar solvents. The optimal conditions arise from combination of ease of formation and stability of the salt, which, in our case, occurred in methanol.

Further optionally, the sulfur-containing compounds are homochiral. By homochiral is meant an enantiomeric species comprising only one enantiomeric form (R or S).

Preferably, the reaction is enantiospecific. By “enantiospecific” is meant capable of synthesising a product having high enantiomeric purity, i.e. that a product comprising a single enantiomer is synthesised. However, it is understood that the product does not have to be exclusively enantiopure, but may be partially enantiopure, and that enantioselective capabilities also fall within the scope of this definition.

Optionally, the reaction is regiospecific. By “regiospecific” is meant being capable of synthesising a product in which one structural isomer is produced in favour of other isomers are also theoretically possible. However, it is understood that the given structural isomer product does not have to be exclusively synthesised, but other structural isomers may be synthesised, and that regioselective capabilities also fall within the scope of this definition.

Optionally, the reaction is stereospecific. By “stereospecific” is meant capable of synthesising products having the same atomic connectivity, wherein a product having a given atomic arrangement in space, or structural configuration, is synthesized in favour of other products of different atomic arrangement in space. However, it is understood that the product having a given atomic arrangement in space does not have to be exclusively synthesised, but products with other structural configuration may be synthesised, and that stereoselective capabilities also fall within the scope of this definition.

Optionally, the reaction is carried out at between −20° C. and 35° C., optionally between −20° C. and 5° C.

Optionally, the reaction is carried out for a period of time such that an isolatable amount of the sulphur-containing compound is produced. Optionally, the period of time is between 20 min and 96 h, optionally between 1 h and 20 h.

The mild conditions adopted in the methods of the present invention allow the preparation of sulfur-containing compounds that contain heat and/or radical sensitive functionalities. As a result, sulfur-containing compounds have been prepared that could not be obtained before using the existing methodology.

Accordingly in a second aspect of the invention, there is provided sulfur-containing compounds of the formula:

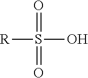

wherein R is selected from:

(a) 1-(4-Nitro-phenyl)-3-oxo-3-phenyl-propane;

(b) 2-(3-Methyl-4-nitro-isoxazol-5-yl)-1-phenyl-ethane;

(c) 1-(4-Methoxy-phenyl)-2-(3-methyl-4-nitro-isoxazol-5-yl)-ethane;

(d) 2-(3-Methyl-4-nitro-isoxazol-5-yl)-1-(4-nitro-phenyl)-ethane;

(e) 1-(4-Fluoro-phenyl)-2-(3-methyl-4-nitro-isoxazol-5-yl)-ethane;

(f) 1-(4-Chloro-phenyl)-2-(3-methyl-4-nitro-isoxazol-5-yl)-ethane; and

(g) 3-Oxo-cyclohexane.

In a third aspect of the invention, there is provided use of heterochiral, optionally homochiral, sulfur-containing compounds for the resolution of racemic mixtures of amines.

Optionally, the heterochiral, optionally homochiral, sulfur-containing compounds are selected from the general formulae 4 and 2:

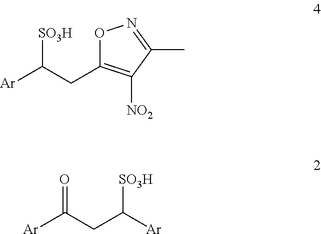

wherein Ar in either of 4 or 2 above is a saturated compound or an unsaturated compound, or a cyclic derivative thereof. Optionally, Ar in either of 4 or 2 above is selected from the group comprising, but not limited to, aromatic compounds, heteroaromatic compounds, and alkyl compounds.

Optionally, the heterochiral, optionally homochiral, sulfur-containing compounds are the heterochiral, optionally homochiral, sulfur-containing compounds obtainable by the method of the first aspect of the invention; or heterochiral, optionally homochiral, sulfur-containing compounds of the second aspect of the invention.

Optionally, the heterochiral, optionally homochiral, sulfur-containing compounds are independently selected from the group comprising molecules 4a-e, or from the group comprising molecules 2a-c. Families of sulfonic acids such as 4a-e could be used to resolve racemic amines (Dutch Resolution). The use of structurally related (families) sulfonic acids has been proved an efficient method to separate two amine enantiomers. However, this methodology is hampered by the limited number of families of homochiral sulfonic acids. The method of the present invention allows the preparation of families of enantiopure sulfonic acids, for example, 4a-e or 2a-c.

Alternatively, the heterochiral, optionally homochiral, sulfur-containing compounds are independently selected from the group comprising molecules 51a and 51b.

It will be appreciated that sulfonic acids prepared in accordance with the method of the present invention can used as surfactants, as synthetic intermediates and/or as resolving agents.

### MATERIALS AND METHODS

### Preparation of Compounds (Example 1)

To a solution of alkene (2.17 mmol, 1 equiv) in THF (10 ml) was added aqueous NaHSO₃(38%) solution (5.95 mL, 21.7 mmol) followed by pyridine (3 mL, 21.7 mmol) at room temperature (RT), and stirred for the number of hours indicated in Table 1. After this time, the salts were filtered, the organic layer separated and evaporated to give the triethylammonium salt of the product in pure form. The salt was then dissolved in water (10 ml) and passed through a DOWEX™ acidic ion exchange resin to eliminate triethylamine. Evaporation of the aqueous layer under reduced pressure at 50° C. gave pure sulfinic acid ester, as a colourless solid.

### Preparation of Compounds (Example 2)

To a solution of alkene (250 mg, 1.0 mmol, 1 equiv) in THF (5 ml) was added aqueous NaHSO₃ (38%) solution (2.47 ml, 10 mmol) followed by TEA (triethylamine) (1.4 ml, 10 mmol) at room temperature, and stirred for 1 h. After this time, the salts were filtered, the organic layer separated and evaporated to give the triethylammonium salt of the product in pure form. The salt was then dissolved in water (10 ml) and passed through a DOWEX™ acidic ion exchange resin to eliminate triethylamine. Evaporation of the aqueous layer under reduced pressure at 50° C. gave pure sulfinic acid ester, as a colourless solid.

### EXAMPLES

The following examples are described herein so as to provide those of ordinary skill in the art with a complete disclosure and description of the invention, and are intended to be purely exemplary of the present invention, and are not intended to limit the scope of the invention.

### Example 1

### Preparation of Sulfonic Acid

During our studies on the dihydroxylation of alkene 3 [Adamo, M. F. A. et Al., Org. Lett. 2008, 10, 1807], we noticed, but did not report, that remainder starting material 3 reacted with sodium bisulfite during work up. This reaction occurred only when an excess of pyridine was present (Table 1). Pyridine was used as the solvent and must be used in at least 5 mL per mmol of alkene. When used in such a large excess, pyridine gives good results.

The reaction with “diluted” pyridine [pyridine/THF, first entry below] is slower (requires 12 h) and gives 60% yield, whilst the “undiluted reaction”[pyridine as solvent, second entry below] requires 3-4 h and gives higher yields. No reaction occurs in the absence of pyridine [last entry below].

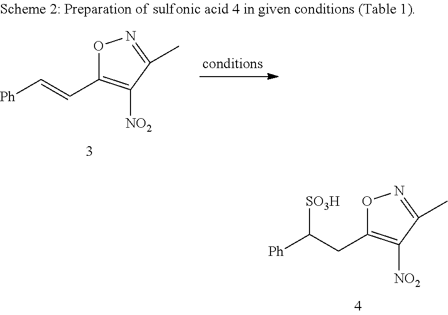

TABLE 1
Yields of sulfonic acid 4.

| Reaction conditions ^{a} | Time (hours) | Yields of 4 ^{b} |
|---|---|---|
| Pyridine/THF, NaHSO₃ | 12 h | 60% |
| Pyridine, NaHSO₃ | 2-3 h | 75% |
| THF, NaHSO₃ | 12 h | 0% |

^{a} reaction conditions:
^{b} yields after flash chromatography, room temperature

TEA, as used in Example 2 below, is thought to be a superior catalyst compared to pyridine, as used herein, because TEA is more capable of engaging in hydrogen-bonding with bisulfite. It is thought that the tighter pair leads to higher reactivity.

### Example 2

### Preparation of Sulfonic Acid Using Different Alkenes

Several alkenes have been submitted to reaction with sodium bisulfite in the presence of pyridine, in first instance, and simple amines such as triethylamine (TEA).

Triethylamine and tertiary amines in general showed to be superior catalysts compared to pyridine and were used in limited amounts. Pyridine is an aromatic amine while triethylamine is not, therefore the lone pair in pyridine is delocalised and less available to engage in hydrogen-bonding (acceptor). In triethylamine, the lone pair is present stably on the nitrogen and therefore a tighter pair could be formed with bisulfite.

Representative results are summarised in Table 2. The reactions in Table 2 were carried out at room temperature (RT) in a very short time, in tetrahydrofuran (THF) as the reaction solvent and without the aid of radical initiators.

We have briefly studied the reaction mechanism and established the following facts:

(a) The conversion of 1 (substrate or acceptor compound) to 2 (sulphur-containing compound) occurred in the absence of light and in the presence of radical scavengers, demonstrating that sulfonic acid 2 arose from thia-Michael addition of bisulfite to the alkene.

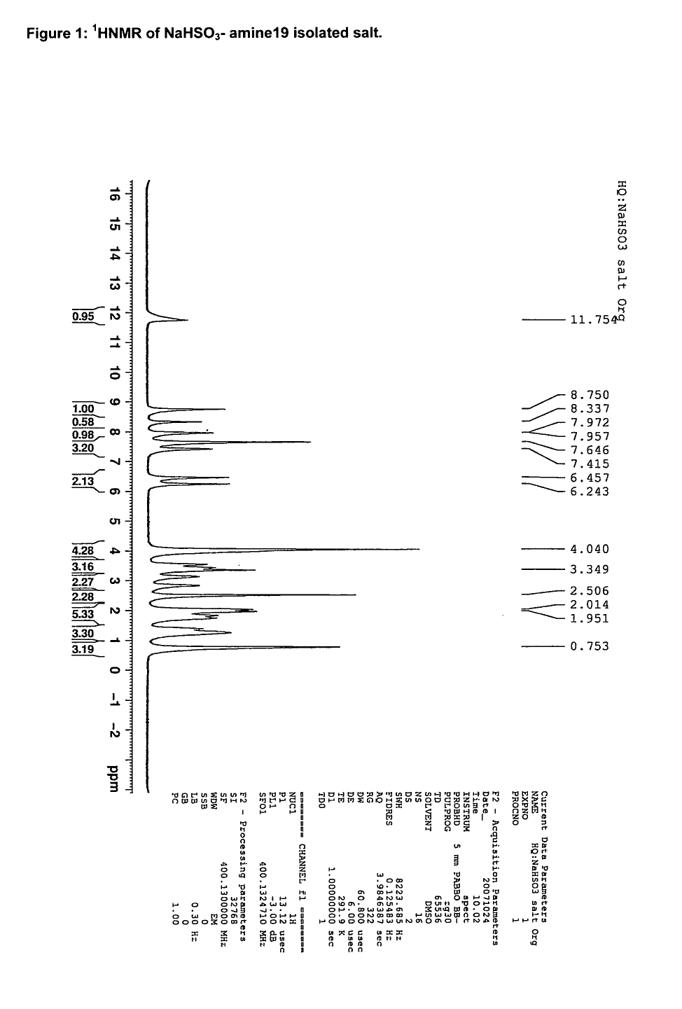

(b) reaction of bisulfite (donor compound) with the amine produce a solid salt that could be isolated (see FIG. 1 of the accompanying drawings). This is one embodiment of the intermediate species. Subsequent treatment of the alkene (acceptor compound) with the salt produced the desired sulfonic acid.

TABLE 2

| | | Amount | Time (hours) and | | |
|---|---|---|---|---|---|
| | Amount of | of TEA | temperature (RT is | | Yield |
| Substrate | NaHSO₃ | (eq is equiv) | room temperature) | Product | % |
| 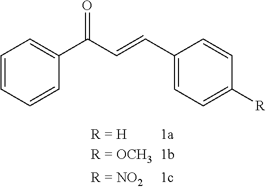 | 10 mol eq | 10 mol eq | 1 h RT | 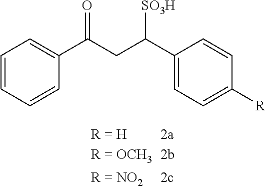 | 2a 93 2b 98 2c 94 |
| 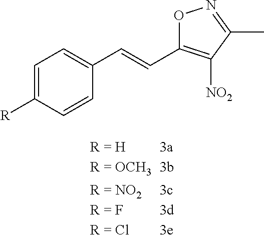 | 10 mol eq | 10 mol eq | 1 h RT | 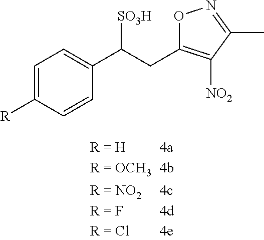 | 4a 75 4b 80 4c 82 4d 86 4e 81 |
| 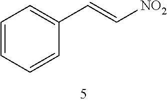 | 10 mol eq | 10 mol eq | 1 h RT | 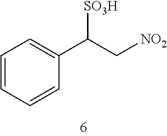 | 92 |
| 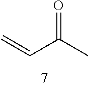 | 1.2 mol eq | 1.2 mol eq | 2 h RT | 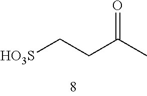 | 88 |
| 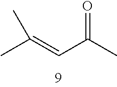 | 1.2 mol eq | 1.2 mol eq | 16 h RT | 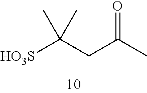 | 82 |
| 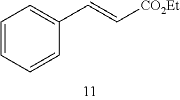 | 20 mol eq | 20 mol eq | 14 h RT | 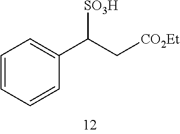 | 71 |
| 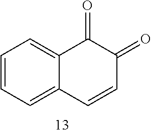 | 10 mol eq | 10 mol eq | 4 h RT | 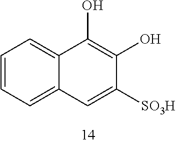 | 79 |
| 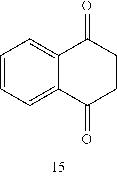 | 10 mol eq | 10 mol eq | 4 h RT | 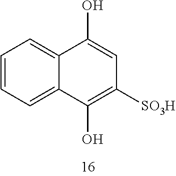 | 84 |
| 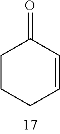 | 1.2 mol eq | 1.2 mol eq | 2 h RT | 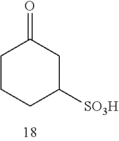 | 91 |
| 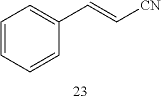 | 20 mol eq | 20 mol eq | 14 h RT | 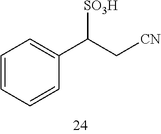 | 89 |
| 23 3-Phenyl-acrylonitrile | | | | | |
| 24 2-Cyano-1-phenyl-ethanesulfonic acid | | | | | |

### Example 3

### Preparation of Sulfonic Acid in Enantiomeric Excess

The reaction of one equiv of 1 (acceptor compound—it will be appreciated that, when the term “equiv” is used, this is a molar equivalent, with respect to one molar equivalent of the substrate) with bisulfite in the presence of 10 equiv of enantiopure hydroquinine 19 and 10 equiv of sodium bisulfite produced 2 in 84% isolated yields and in 33% enantiomeric excess. The use of amines 20 and 21 (illustrated in Scheme 3 below) furnished 2 in similar yields and in 11-15% enantiomeric excess. Importantly, in these experiments, the amine was recovered at the end of the reaction. To date, this is the unique example of asymmetric sulfonylation observed.

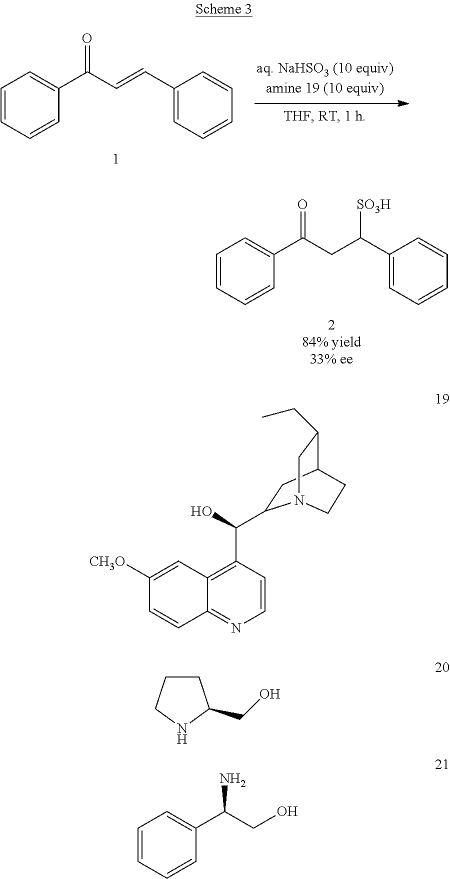

To a solution of chiral amine 19 or 19a from Scheme 4 (1.1 mmol) in MeOH (10 mL) was added aq. NaHSO₃ (38%) solution dropwise (0.32 mL, 1.1 mmol). The solution was stirred for one hour at room temperature, then cooled to 0-5° C. and stirred for 30 minutes, treated with a solution of alkene (1.0 equiv, 1.0 mmol) in THF (10 mL). The reaction mixture was then stirred for 12 hours at room temperature. After this time, the solvents were evaporated to dryness, the solid obtained were treated with chloroform (15 mL) and filtered. The chloroform layer was evaporated and the crude was analysed by ¹H-NMR and weighed.

Catalyst 19a could be used in just 1.1 equiv ensuring 100% conversion of alkene 1 and over 99% yield of desired 2. Importantly, catalyst 19a allowed the preparation of sulfonic acid 2 in 86% enantiomeric excess (Scheme 4 and Table 3).

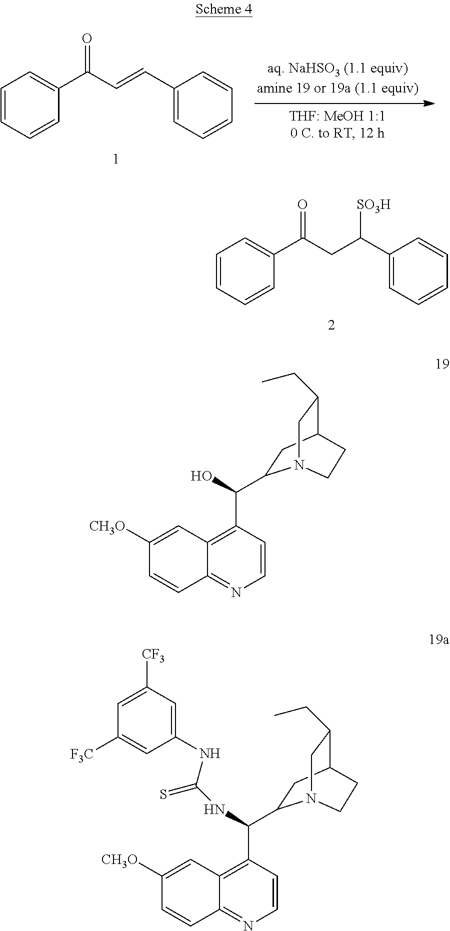

TABLE 3

| Amine | NaHSO₃ | Yields % 2 | ee % |
|---|---|---|---|
| NEt₃ (1.1 equiv) | 1.1 equiv | 30% | 0 |
| 19 (1.1 equiv) | 1.1 equiv | 50% | 30% |
| 19a (1.1 equiv) | 1.1 equiv | >99% | 86% |

Without being bound by theory, we postulate that activation of bisulfite occurs through formation of H-bonds. We also have proposed that the higher turnover of catalyst 25 (Scheme 5) over triethylamine, depends on the ability of compound 25 to form two H-bonds with bisulfite, while triethylamine could make only one such H-bond.

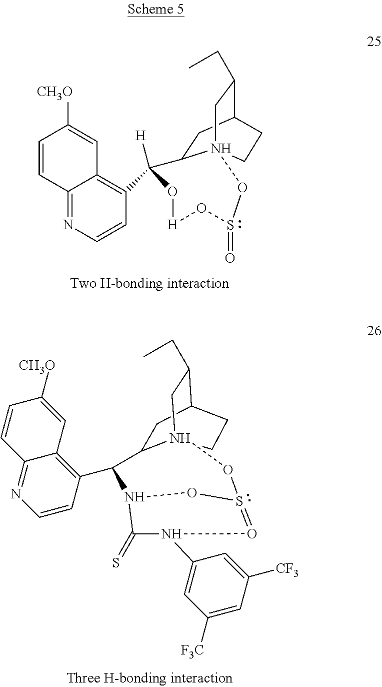

It is reasoned that the use of catalysts capable of three H-bonds, such as thiourea 26, should impart to bisulfite, enhanced reactivity (Scheme 5). The concept was proved true and the catalyst 26 could be used in just 1.1 equiv, ensuring 100% conversion of alkene 1a-c of Table 2 and over 99% yield of the desired 2a-c. Importantly, catalyst 26 allowed the preparation of sulfonic acid 2a-c in 86-88% enantiomeric excess. Thioureas, such as thiourea 26, are preferred catalysts.

The NaHSO₃— amine isolated salt (intermediate compound) 22, in which the bisulfite is in hydrogen bonding interaction with catalyst 19, is characterised by ¹HNMR in FIG. 1 of the accompanying drawings.

### Example 4

### Procedure for the Addition of Bisulfite to Epoxides or Aziridines

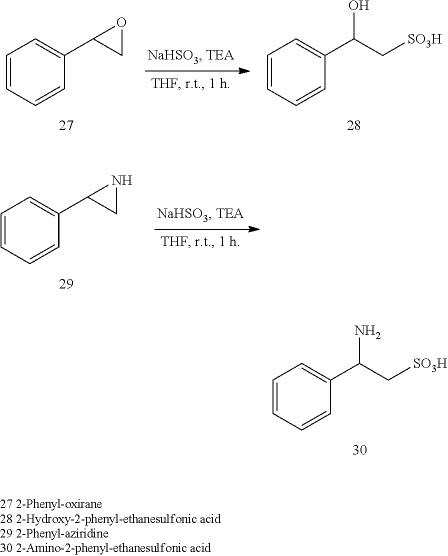

To a solution of amine (1.1 mmol) in MeOH (10 mL) was added aq. NaHSO₃(38%) solution dropwise (0.32 mL, 1.1 mmol). The solution was stirred for one hour at room temperature, then cooled to 0-5° C. and stirred for 30 minutes, treated with a solution of epoxide 26 or aziridine 28 (1.0 equiv, 1.0 mmol) in THF (10 mL). The reaction mixture was then stirred for 12 hours at room temperature. After this time, the solvents were evaporated to dryness, the solid obtained were treated with chloroform (15 mL) and filtered. The chloroform layer was evaporated and the crude was analysed by ¹H-NMR and weighed.

Yield for 28 169 mg, 84% yield

Yield for 30 163 mg, 81% yield

### Example 5

### Preparation of Sulfonic Acid in Enantiomeric Excess Using Substoichiometric (Catalytic) Amounts of Amines

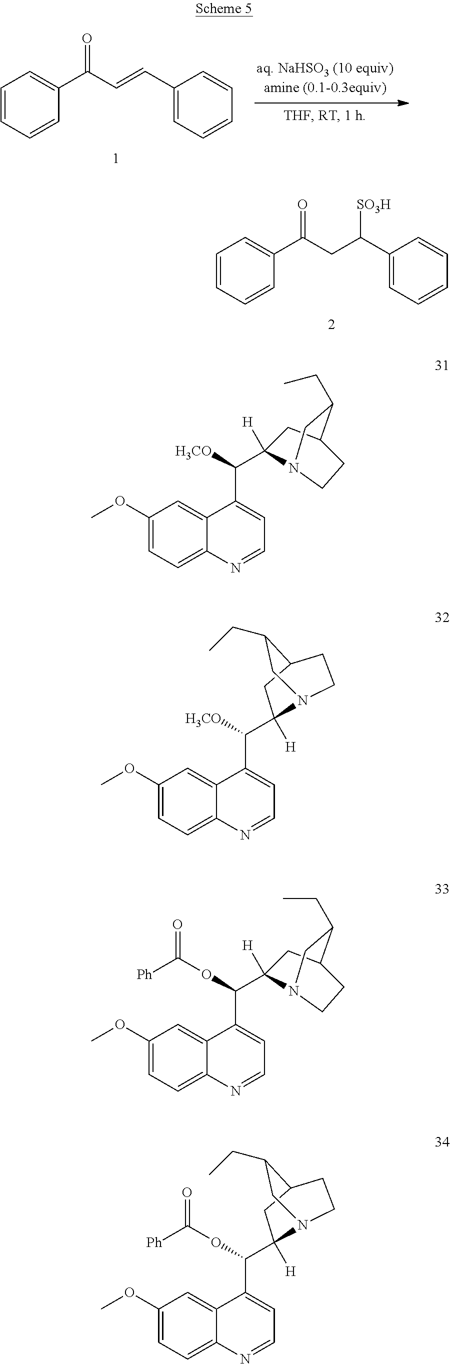

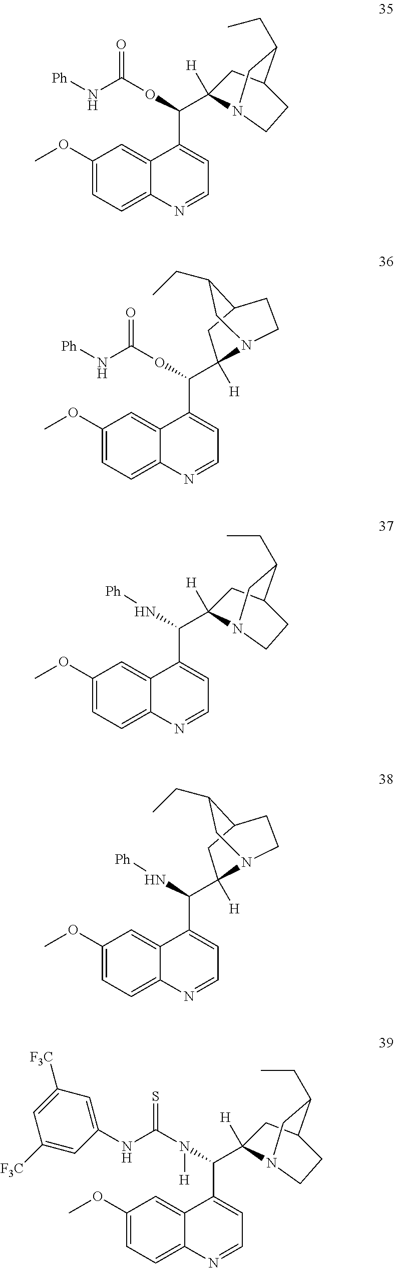

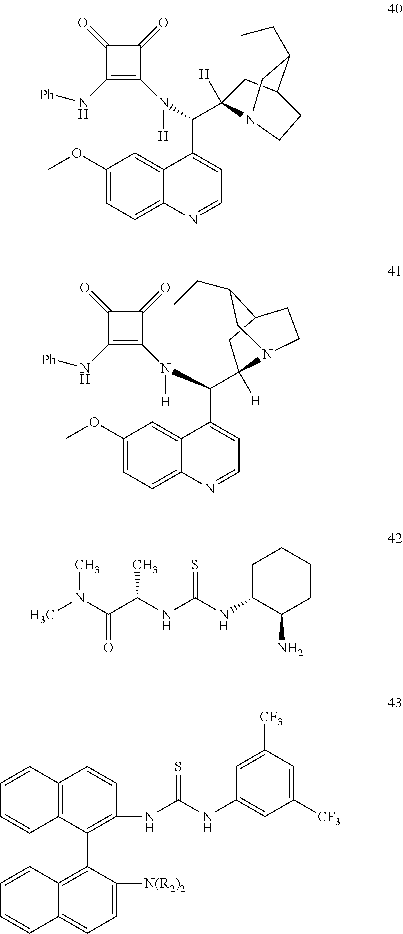

To a solution of chiral amine, each independently selected from molecules 31-43 above and of Table 4a, (0.1 mmol-0.3 equiv) in MeOH (5 mL) and THF (5 mmol) were added sequentially aq. NaHSO₃ (38%) solution (3.2 mL, 10 equiv) and alkene 1 (1.0 equiv, 1 mmol). The reaction mixture was then stirred for 96 hours at room temperature. After this time, the solvents were evaporated to dryness, the solid obtained were treated with chloroform (15 mL) and filtered. The chloroform layer was evaporated and the crude was analysed by ¹H-NMR and weighed. Yields and enantiomeric excesses obtained using 0.1 equiv or 0.2 or 0.3 equiv of catalyst gave identical yields and enantiomeric purities.

TABLE 4a

| Amine | NaHSO₃ | Yield % 2 | ee % |
|---|---|---|---|
| 31 | 10 equiv | 80% | 33% |
| 32 | 10 equiv | 85% | 30% |
| 33 | 10 equiv | 89% | 25% |
| 34 | 10 equiv | 84% | 35% |
| 35 | 10 equiv | 86% | 36% |
| 36 | 10 equiv | 89% | 31% |
| 37 | 10 equiv | 95% | 42% |
| 38 | 10 equiv | 96% | 45% |
| 39 | 10 equiv | 96% | |
| 40 | 10 equiv | 92% | |
| 41 | 10 equiv | 94% | |
| 42 | 10 equiv | 91% | |
| 43 | 10 equiv | 95% | |

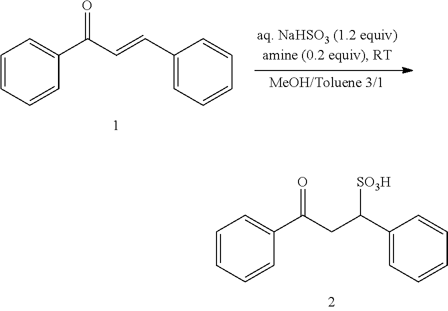

To a solution of alkene 1 (0.2 mmol, 1 equiv) in MeOH/Toluene 3/1 (2 mL) was added aqueous NaHSO₃ (0.5M) (0.5 mL, 1.2 equiv) solution followed by amine (0.2 equiv) (each independently selected from molecules 31-43 of Table 4b) and stirred at room temperature for 16 h. After this time the reaction mixture was evaporated, the solids obtained taken up in THF/water (1/1, 3×3 mL) and the solution passed through activated DOWEX resin. The sulfonic acid was discharged from DOWEX by washing with water (3×3 mL) which was the evaporated to give colourless solid. Yields and enantiomeric excesses obtained using 0.1 equiv or 0.2 or 0.3 equiv of catalyst gave identical yields and enantiomeric purities.

TABLE 4b

| Amine | Yield % 2 | ee % |
|---|---|---|
| 31 | 80% | 33% |
| 32 | 85% | 30% |
| 33 | 89% | 25% |
| 34 | 84% | 35% |
| 35 | 86% | 36% |
| 36 | 89% | 31% |
| 37 | 95% | 42% |
| 38 | 96% | 45% |
| 39 | 96% | 91% |
| 40 | 92% | 38% |
| 41 | 94% | 5% |
| 42 | 91% | 0% |
| 43 | 95% | 30% |

In relation to amines 39-43, there is no great variation of yields and ees with changes in reaction condition, because amines 31-38 are relatively poor as asymmetric catalysts. On the contrary, amine 39 is very good and gives results similar enough to amine 19a which is the currently preferred catalyst.

### Example 6

### Preparation of Sulfonic Acid in Enantiomeric Excess Using Substoichiometric (Catalytic) Amounts of Ammoniums Salts as Phase Transfer Catalysts

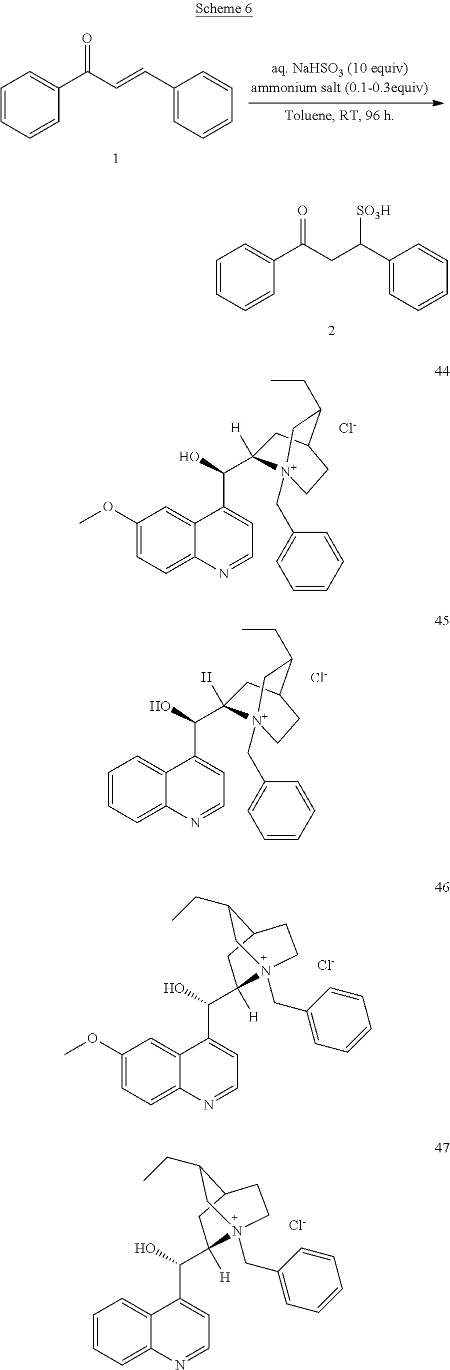

TABLE 5a

| Amine | NaHSO₃ | Yields % 2 | ee % |
|---|---|---|---|
| 44 | 10 equiv | 96% | 75% |
| 45 | 10 equiv | 92% | 86% |
| 46 | 10 equiv | 94% | 88% |
| 47 | 10 equiv | 91% | 89% |

To a solution of chiral ammonium salt, each independently selected from molecules 44-47 of Table 5a, (0.1 mmol-0.3 mmol) in Toluene (5 mL) were sequentially added aq. NaHSO₃(38%) solution (3.2 mL, 10 mmol) and alkene 1 (1.0 equiv, 1.0 mmol). The reaction mixture was then stirred for 96 hours at room temperature. After this time, the solvents were evaporated to dryness, the solid obtained were treated with chloroform (15 mL) and filtered. The chloroform layer was evaporated the sulfonic acid obtained after purification using a Dowex resin, the crude weighted, analysed by ¹H-NMR and the enantiomeric excess registered by chiral HPLC run on the methylsulfonate ester. Yields and enantiomeric excesses obtained using 0.1 equiv or 0.2 or 0.3 equiv of catalyst gave identical yields and enantiomeric purities.

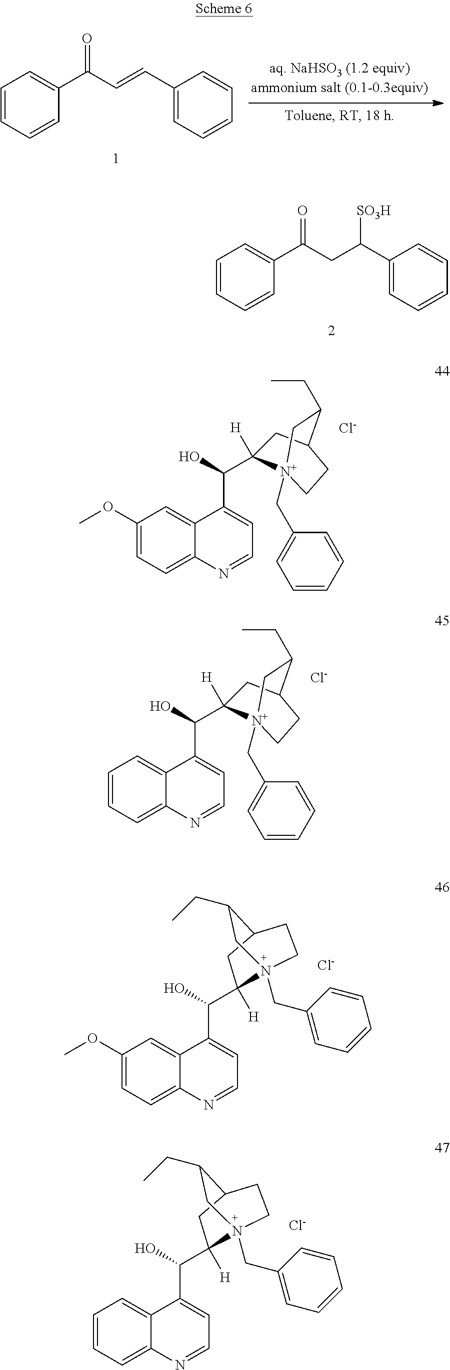

TABLE 5b

| Amine | NaHSO₃ | Yields % 2 | ee % |
|---|---|---|---|
| 44 | 1.2 equiv | 96% | 75% |
| 45 | 1.2 equiv | 92% | 86% |
| 46 | 1.2 equiv | 94% | 88% |
| 47 | 1.2 equiv | 91% | 89% |

To a solution of chiral ammonium salt, each independently selected from molecules 44-47 of Table 5b, (0.1 mmol-0.3 mmol) in Toluene (5 mL) were sequentially added aqueous NaHSO₃ (0.5M) (0.5 mL, 1.2 equiv) solution and alkene 1 (1.0 equiv, 1.0 mmol). The reaction mixture was then stirred for 18 hours at room temperature. After this time, the solvents were evaporated to dryness, the solid obtained were treated with chloroform (15 mL) and filtered. The chloroform layer was evaporated the sulfonic acid obtained after purification using a Dowex resin, the crude weighted, analysed by ¹H-NMR and the enantiomeric excess registered by chiral HPLC run on the methylsulfonate ester. Yields and enantiomeric excesses obtained using 0.1 equiv or 0.2 or 0.3 equiv of catalyst gave identical yields and enantiomeric purities.

The yields and % ee are the same in each of Tables 5a and 5b because the reaction has two phases (toluene and water), each containing one of the reactants, hence there is no background reaction. The ammonium salt 44-47 is thought to be operating as a shuttle catching one reagent in the water phase and bringing it in the organic layer. Therefore, it is only the bisulfite present in toluene that must be bound to the catalyst.

### Example 7

### Addition of Sodium Bisulfite to Imines: Preparation of Enantiopure Alpha-Aminosulfonic Acids

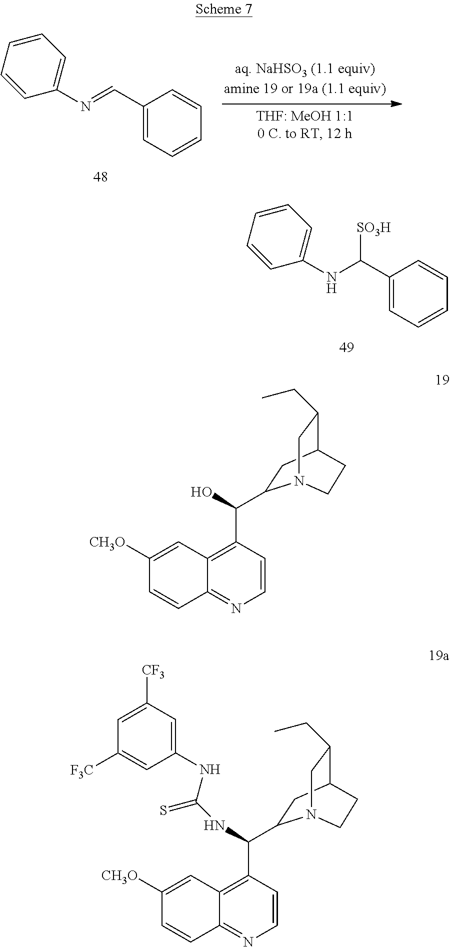

To a solution of NEt₃ or amines 19-19a (all at 1.1 mmol) in MeOH (10 mL) was added aq. NaHSO₃ (38%) solution dropwise (0.32 mL, 1.1 mmol). The solution was stirred for one hour at room temperature, then cooled to 0-5° C. and stirred for 30 minutes, treated with a solution of imine (1.0 equiv, 1 mmol) in THF (10 mL). The reaction mixture was then stirred for 12 hours at room temperature. After this time, the solvents were evaporated to dryness, the solid obtained were treated with chloroform (15 mL) and filtered. The chloroform layer was evaporated and the crude was analysed by ¹H-NMR and weighed.

TABLE 6a

| Amine | NaHSO₃ | Yields % 49 | ee % |
|---|---|---|---|
| NEt₃ (1.1 equiv) | 1.1 equiv | 30% | 0 |
| 19 (1.1 equiv) | 1.1 equiv | 50% | 25% |
| 19a (1.1 equiv) | 1.1 equiv | >99% | 90% |

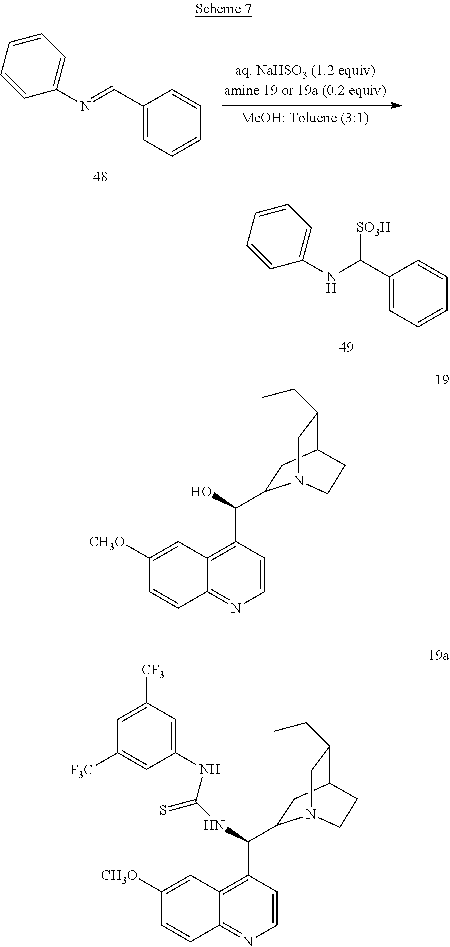

To a solution of imine 48 (0.2 mmol, 1 equiv) and NEt₃ or amines 19-19a (0.2 equiv) in MeOH Toluene 3:1 (2 mL) was added NaHSO₃ (0.5M) (0.5 mL, 1.2 equiv). The solution was stirred for 6 h at room temperature. After this time, the solvents were evaporated to dryness, the solid obtained were treated with chloroform (15 mL) and filtered. The chloroform layer was evaporated and the crude was analysed by ¹H-NMR and weighed.

TABLE 6b

| Amine | NaHSO₃ | Yield % 49 | ee % |
|---|---|---|---|
| NEt₃ (0.2 equiv) | 1.1 equiv | 95% | 0 |
| 19 (0.2 equiv) | 1.2 equiv | 95% | 25% |
| 19a (0.2 equiv) | 1.2 equiv | >99% | 95% |

The results in Table 6b for the amine, triethylamine, is a faster reaction and requires only 0.2 equiv. of amine, which qualifies this for being a catalytic reaction. The results in Table 6a are the same in terms of yields and ees but they required 1.1-1.2 equiv of amine which was, in this case, being consumed as a reagent and not, strictly speaking, a catalyst.

As used in this specification, the term “catalyst” is intended to embrace entities that assist in accelerating a reaction rate whether they are consumed in the reaction, as well as, entities that act as a “true” catalyst and are regenerated during the reaction.

### Example 8

### Addition of Sodium Bisulfite to Aldehydes or Ketones: Preparation of Alpha-Methoxysulfonic Acids

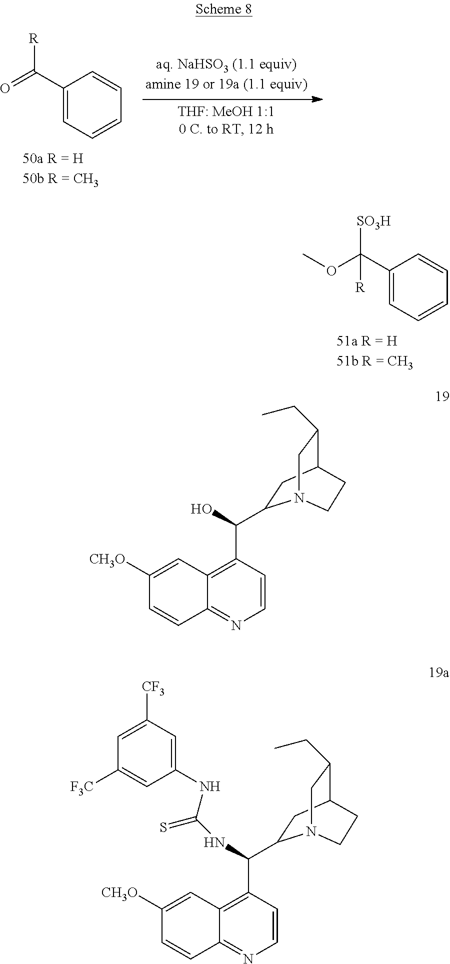

To a solution of NEt₃ or amines 19-19a (1.1 mmol) in MeOH (10 mL) was added aq. NaHSO₃ (38%) solution dropwise (0.32 mL, 1.1 mmol). The solution was stirred for one hour at room temperature, then cooled to 0-5° C. and stirred for 30 minutes, treated with a solution of aldehyde 50a or ketone 50b (1.0 equiv, 1.0 mmol) in THF (10 mL). The reaction mixture was then stirred for 12 hours at room temperature. After this time, acetylchloride (0.5 equiv) was added and the reaction mixture was stirred for further 5 h. The solvents were evaporated to dryness, the solid obtained were treated with chloroform (15 mL) and filtered. The chloroform layer was evaporated and the crude was analysed by ¹H-NMR and weighed.

TABLE 7a

| Amine | R | NaHSO₃ | Yields % 51 | ee % |
|---|---|---|---|---|
| NEt₃ (1.1 equiv) | H | 1.1 equiv | 30% | 0 |
| NEt₃ (1.1 equiv) | CH₃ | 1.1 equiv | 25% | 0 |
| 19 (1.1 equiv) | H | 1.1 equiv | 50% | 25% |
| 19 (1.1 equiv) | CH₃ | 1.1 equiv | 92% | 85% |
| 19a (1.1 equiv) | H | 1.1 equiv | >99% | 90% |
| 19a (1.1 equiv) | CH₃ | 1.1 equiv | 96% | 96% |

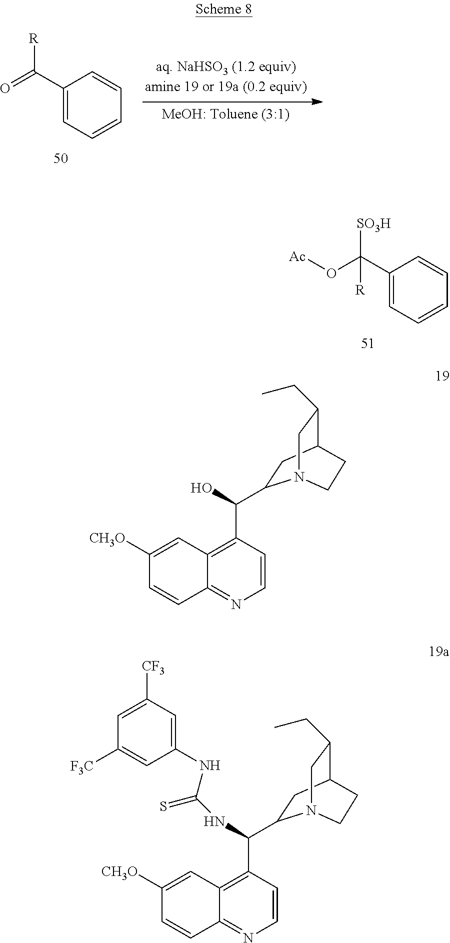

To a solution of carbonyl compound 50 (R═H or CH₃) (0.2 mmol) and NEt₃ or amines 19-19a (0.2 equiv) in MeOH:Toluene 3:1 (2 mL) was added NaHSO₃ (0.5M) (0.5 mL, 1.2 equiv). The solution was stirred for 6 h at room temperature. After this time, acetylchloride (1.0 equiv, 1.0 mmol) was added and the reaction mixture was stirred for further 1 h. The solvents were evaporated to dryness, the solid obtained were treated with chloroform (15 mL) and filtered. The chloroform layer was evaporated and the crude was analysed by ¹H-NMR and weighed.

TABLE 7b

| Amine | R | NaHSO₃ | Yields % 51 | ee % |
|---|---|---|---|---|
| NEt₃ (1.1 equiv) | H | 1.2 equiv | 30% | 0 |
| NEt₃ (1.1 equiv) | CH₃ | 1.2 equiv | 25% | 0 |
| 19 (0.2 equiv) | H | 1.2 equiv | 50% | 25% |
| 19 (0.2 equiv) | CH₃ | 1.2 equiv | 92% | 85% |
| 19a (0.2 equiv) | H | 1.2 equiv | >99% | 90% |
| 19a (0.2 equiv) | CH₃ | 1.1 equiv | 96% | 96% |

### Example 9

### Addition of Sulfinic Acid to Alkenes: Preparation of Enantiopure Sulfones

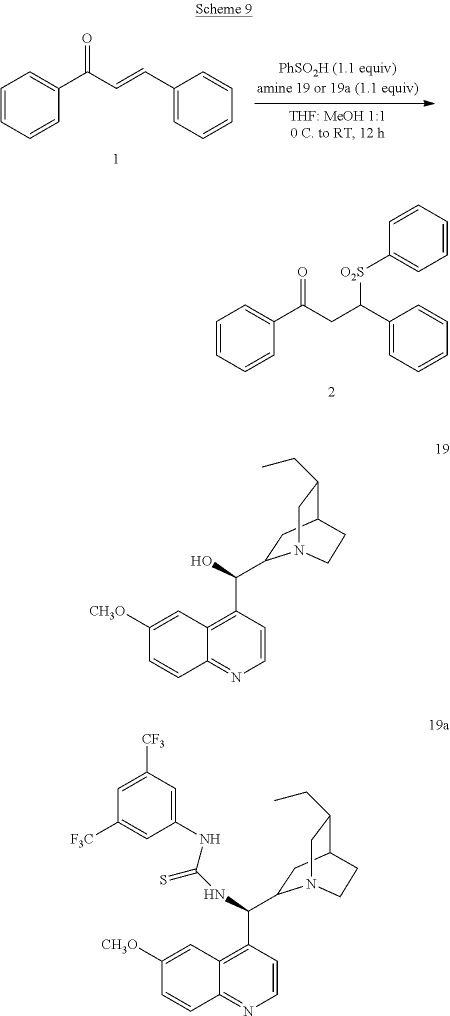

To a solution of NEt₃ or amines 19-19a (all at 1.1 equiv) in MeOH (10 mL) was added Phenylsulfinic acid (1.1 equiv). The solution was stirred for one hour at room temperature, then cooled to 0-5° C. and stirred for 30 minutes, treated with a solution of alkene 1 (1.0 equiv, 1.0 mmol) in THF (10 mL). The reaction mixture was then stirred for 12 hours at room temperature. After this time, the solvents were evaporated to dryness, the solid obtained were treated with chloroform (15 mL) and filtered. The chloroform layer was evaporated and the crude was analysed by ¹H-NMR and weighed.

TABLE 8

| Amine | NaHSO₃ | Yields % 2 | ee % |
|---|---|---|---|
| NEt₃ (1.1 equiv) | 1.1 equiv | 90% | 0 |
| 19 (1.1 equiv) | 1.1 equiv | 96% | 56% |
| 19a (1.1 equiv) | 1.1 equiv | >99% | 96% |

### Example 10

### Addition of Sulfenic Acid to Alkynes: Preparation of Enantiopure Sulfoxides

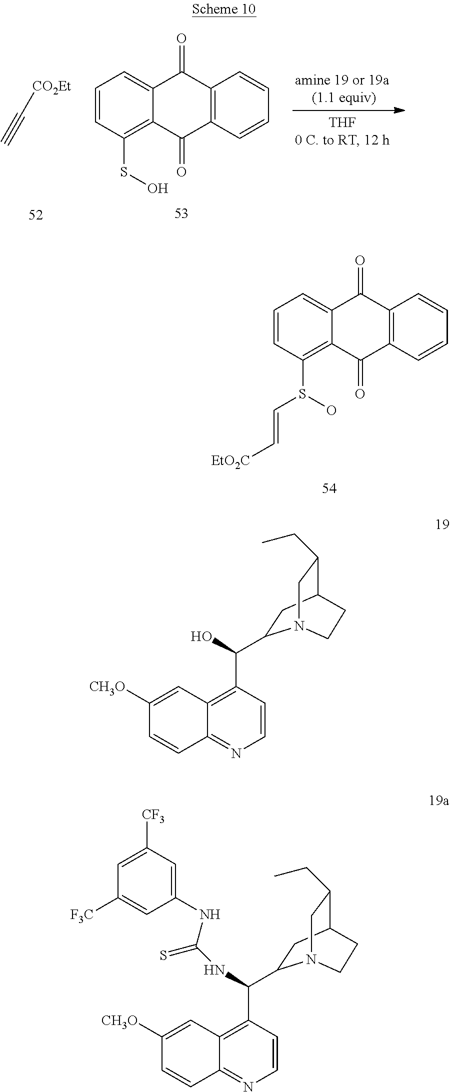

To a solution of NEt₃ or amines 19-19a (all at 1.1 equiv) in THF (10 mL) were sequentially added anthraquinone sulfenic acid 53 (1.1 equiv) and ethyl propiolate 52 (1.0 equiv, 1.0 mmol) in THF (10 mL). The reaction mixture was then stirred for 6 hours at room temperature. After this time, the solvents were evaporated to dryness, the solid obtained were treated with chloroform (15 mL) and filtered. The chloroform layer was evaporated and the crude was analysed by ¹H-NMR and weighed to give compound 54 in yields and enantiomeric excess reported in table 9.

TABLE 9

| Amine | NaHSO₃ | Yields % 54 | ee % |
|---|---|---|---|
| NEt₃ (1.1 equiv) | 1.1 equiv | 90% | 0 |
| 19 (1.1 equiv) | 1.1 equiv | 96% | 56% |
| 19a (1.1 equiv) | 1.1 equiv | >99% | 96% |

### Example 11

### Preparation of Compounds

To a solution of acceptor compound (0.2 mmol, 1 equiv) in MeOH (2 mL) was added, aqueous NaHSO₃ (0.5M) (0.5 mL, 1.2 equiv) solution followed by triethylamine (20 mg, 0.04 mmol, 0.2 equiv) and stirred at room temperature for the time specified in Table 10. After the specified time, the reaction mixture was evaporated, the solids obtained taken up in THF/water (1/1, 3×3 mL) and the solution passed through activated DOWEX™ resin. The sulfonic acid was discharged from DOWEX by washing with water (3×3 mL) which was the evaporated to give a colourless solid.

TABLE 10
catalytic racemic sulfonylation using NEt₃
In the reaction scheme below, EWG stands for electron withdrawing group and R stands for a
general organic residue.

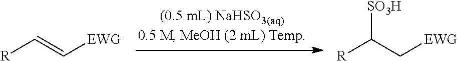

| | | | NaHSO₃ | | | | | |
|---|---|---|---|---|---|---|---|---|
| | | | (aq) | | | | Conv.^{a} | Yield |
| Entry | Acceptor Compound | Product | 0.5 M | Et₃N | Temp. | time | (%) | (%) |
| 1 | 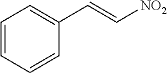 | 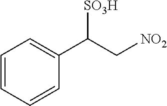 | 1.2 eq. | 0.2 eq. | 22° C. | 10 min | >98 | 70 |
| 2 | 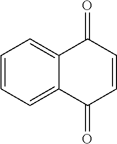 | 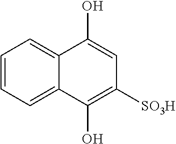 | 1.2 eq. | 0.2 eq. | 22° C. | 10 min | >98 | 73 |
| 3 | 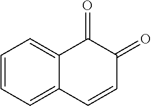 | 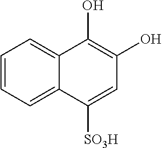 | 1.2 eq. | 0.2 eq. | 22° C. | 10 min | >98 | 71 |
| 4 | 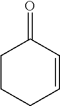 | 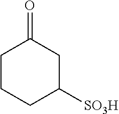 | 1.2 eq. | 0.2 eq. | 22° C. | 21 h | >98 | 89 |
| 5 | 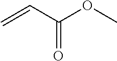 | 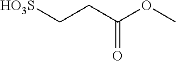 | 1.2 eq. | 0.2 eq. | 22° C. | 39 h | >98 | 85 |
| 6 | 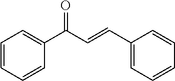 | 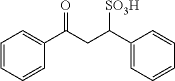 | 1.2 eq. | 0.2 eq. | 22° C. | 21 h | >98 | 93 |
| 7 | 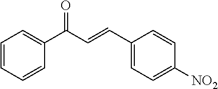 | 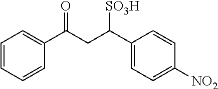 | 1.2 eq. | 0.2 eq. | 22° C. | 39 h | >98 | 95 |
| 8 | 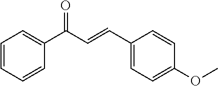 | 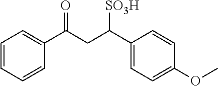 | 1.2 eq. | 0.2 eq. | 22° C. | 48 h | >98 | 91 |
| 9 | 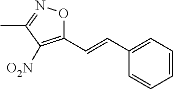 | 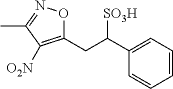 | 1.2 eq. | 0.2 eq. | 22° C. | 24 h | >98 | 94 |
| 10 | 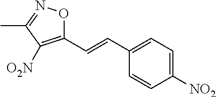 | 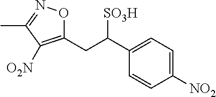 | 1.2 eq. | 0.2 eq. | 22° C. | 48 h | >98 | 96 |
| 11 | 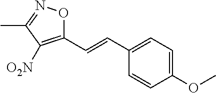 | 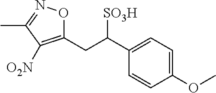 | 1.2 eq. | 0.2 eq. | 22° C. | 72 h | >98 | 91 |
| 12 | 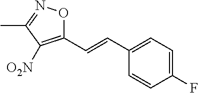 | 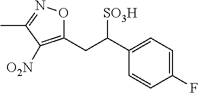 | 1.2 eq. | 0.2 eq. | 22° C. | 24 h | >98 | 96 |
| 13 | 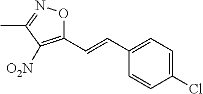 | 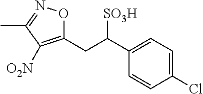 | 1.2 eq. | 0.2 eq. | 22° C. | 24 h | >98 | 91 |
| 14 | 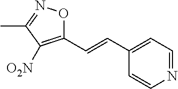 | 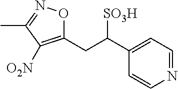 | 1.2 eq. | 0.2 eq. | 22° C. | 24 h | >98 | 94 |
| 15 | 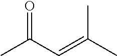 | 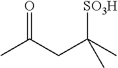 | 1.2 eq. | 0.2 eq. | 22° C. | 18 h | <30 | 23 |
| 16 | 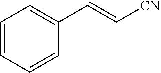 | 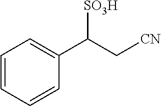 | 1.2 eq. | 0.2 eq. | 22° C. | 72 h | <30 | 21 |
| 17 | 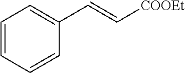 | 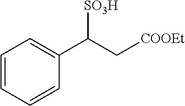 | 1.2 eq | 0.2 eq. | 22° C. | 72 h | <30 | 18 |
| 18 | 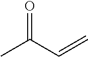 | 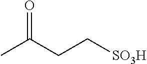 | 1.2 eq | 0.2 eq. | 22° C. | 72 h | <30 | 15 |
| 19 | 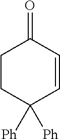 | 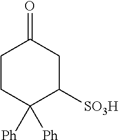 | 1.2 eq | 0.2 eq. | 22° C. | 72 h | <30 | 17 |

^{a}Reaction stopped upon disappearance of starting material.

The compounds in Table 11 (below) reacted only at high temperature (reflux) and after prolonged time. The reactions in Table 11 did not proceed without amine, however they are not catalytic

TABLE 11
stoichiometric sulfonylation using NEt₃

| | | | NaHSO₃ | | | | | |
|---|---|---|---|---|---|---|---|---|
| | | | (aq) | | | | Conv.^{a} | Yield |
| Entry | Acceptor Compound | Product | 0.5 M | Et₃N | Temp. | time | (%) | (%) |
| 1 | 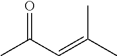 | 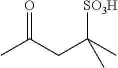 | 1.2 eq. | 1.2 eq. | reflux | 18 h | >98 | 98% |
| 2 | 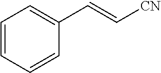 | 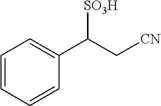 | 5 eq. | 5 eq. | reflux | 4d | >98 | 96 |
| 3 | 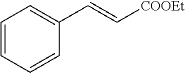 | 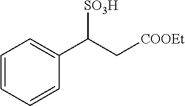 | 5 eq. | 5 eq. | reflux | 4d | >98 | 91 |
| 4 | 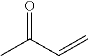 | 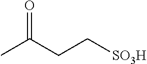 | 5 eq. | 5 eq. | reflux | 4d | >98 | 96 |
| 5 | 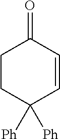 | 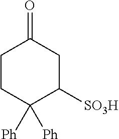 | 10 eq. | 10 eq. | reflux | 4d | >98 | 93 |

TABLE 12
Acceleration of reaction rate with diluted bisulfite solutions

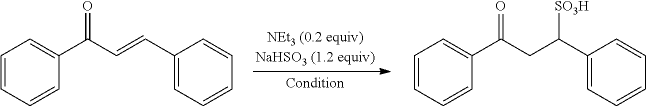

| | | | | | | Time to | App |
|---|---|---|---|---|---|---|---|
| | | | | | | 100% | rate |
| Entry | MeOH | NaHSO₃ aq. | Vₜₒₜ | [NaHSO₃] | NaHSO₃ | conv. | mmol/h |
| 1 | 2 mL | 0.050 mL (4.8 M) | 2.05 mL | 0.117 M | 1.2 equiv | 72 h | 0.0016 |
| 2 | 2 mL | 0.060 mL (3.8 M) | 2.06 mL | 0.116 M | 1.2 equiv | 62 h | |
| 3 | 2 mL | 0.070 mL (3.5 M) | 2.07 mL | 0.115 M | 1.2 equiv | 51 h | |
| 4 | 2 mL | 0.080 mL (3.0 M) | 2.08 mL | 0.114 M | 1.2 equiv | 40 h | |
| 5 | 2 mL | 0.100 mL (2.5 M) | 2.10 mL | 0.114 M | 1.2 equiv | 32 h | |
| 6 | 2 mL | 0.120 mL (2.0 M) | 2.12 mL | 0.113 M | 1.2 equiv | 21 h | |
| 7 | 2 mL | 0.160 mL (1.5 M) | 2.16 mL | 0.111 M | 1.2 equiv | 12 h | |
| 8 | 2 mL | 0.240 mL (1.0 M) | 2.24 mL | 0.107 M | 1.2 equiv | 6 h | |
| 9 | 2 mL | 0.500 mL (0.48 M) | 2.5 mL | 0.096 M | 1.2 equiv | 2 h | 0.06 |
| 10 | 2 mL | 2.4 mL (0.25 M) | 4.4 mL | 0.054 M | 1.2 equiv | 20 h | |

Table 12 shows there is a remarkable acceleration of the reaction rate when diluted (0.48M) NaHSO₃ was employed as the reagent, even though the same absolute amount of donor compound (NaHSO₃) is present (1.2 equiv). The time required to attain complete conversion went from 72 h, using a 4.8M solution of NaHSO₃, to just 2 h when a 0.48M solution was employed. (Compare entries 1 and 9, there is a 36-fold increase of apparent rate with dilution). The fact that a higher reaction rate was observed upon using the same amount of a diluted reagent is counter intuitive.

### Example 12

### Preparation of Sulfonic Acid in Enantiomeric Excess

We have a set of optimised conditions that allow high conversion and high enantiomeric excess to be obtained using down to 0.05 equiv of chiral amine 19a (entry 5). The best condition employs 0.1 equiv. of amine, 1.2 equiv of bisulfite to give compound 2 in 95% yield and 95% enantiomeric excess. This is a truly catalytic and asymmetric process.

TABLE 13
Optimisation of enantioselective catalytic sulfonylation

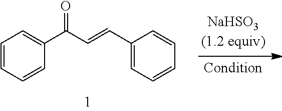

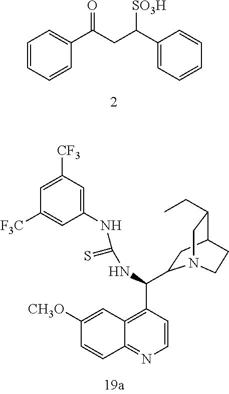

| | | Catalyst | NaHSO₃ | | Temp | Time | Yield | Ee |
|---|---|---|---|---|---|---|---|---|
| Entry | Cat | (equiv) | _{(aq)} | Solvent | ° C. | (h) | 2 (%) | (%) |
| 1 | 19a | 0.2 | 4.8 M | MeOH/ | 22 | 96 | 95 | 40 |
| | | | | Toluene | | | | |
| | | | | 3/1 | | | | |
| 2 | 19a | 0.2 | 0.48 M | MeOH/ | 22 | 2 | 94 | 93 |
| | | | | Toluene | | | | |
| | | | | 3/1 | | | | |
| 3 | 19a | 0.2 | 0.48 M | MeOH/ | -2 | 18 | 95 | 96 |
| | | | | Toluene | | | | |
| | | | | 3/1 | | | | |
| 4 | 19a | 0.1 | 0.48 M | MeOH/ | -2 | 40 | 95 | 95 |
| | | | | Toluene | | | | |
| | | | | 3/1 | | | | |
| 5 | 19a | 0.05 | 0.48 | MeOH/ | 22 | 18 | 40 | 89 |
| | | | | Toluene | | | | |
| | | | | 3/1 | | | | |
| 5 | 19a | 0.2 | 0.48 | MeOH/ | 22 | 18 | 96 | 82 |
| | | | | CH₂Cl₂ | | | | |
| | | | | 1/1 | | | | |
| 6 | 19a | 0.2 | 0.48 | MeOH/ | 22 | 18 | 96 | 95 |
| | | | | Toluene | | | | |
| | | | | 1/1 | | | | |

Reaction scope: the condition highlighted in Table 13, entry 4 were used to prepare the compounds in Table 2 in high enantiomeric excesses.

TABLE 14
Scope of reaction.

| | | | | | After |
|---|---|---|---|---|---|
| | | | Yield | Ee | Crysta |
| Entry | Reactant or Acceptor Compound | Product | % | % | II |
| 1 | 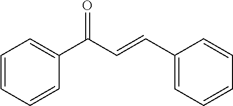 | 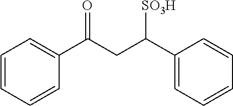 | 95 | 96 | 99.9 |
| 2 | 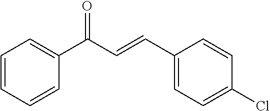 | 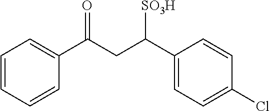 | 92 | 95 | 99.9 |
| 3 | 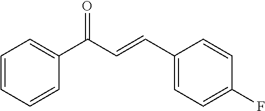 | 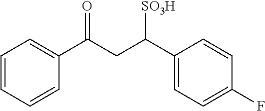 | 90 | 96 | 99.9 |
| 4 | 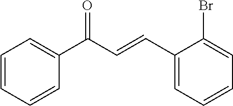 | 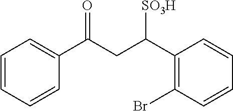 | 86 | 92 | 99.9 |
| 5 | 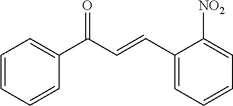 | 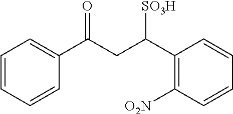 | 92 | 93 | 99.9 |
| 6 | 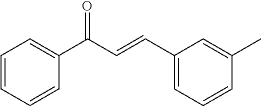 | 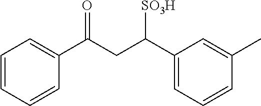 | 93 | 97 | 99.9 |
| 7 | 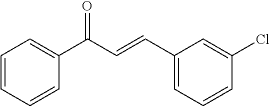 | 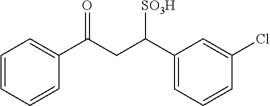 | 90 | 96 | 99.9 |
| 8 | 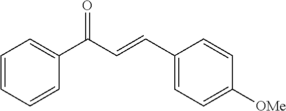 | 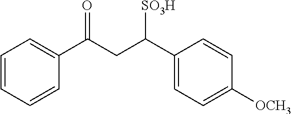 | 89 | 96 | 99.9 |
| 9 | 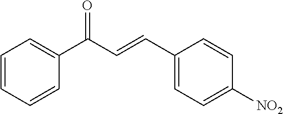 | 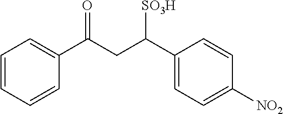 | 93 | 94 | 99.9 |
| 10 | 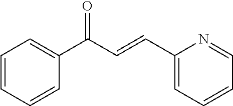 | 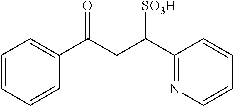 | 94 | 92 | 99.9 |
| 11 | 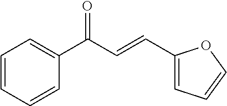 | 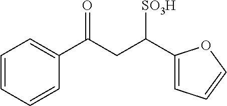 | 75 | 84 | 99.9 |
| 12 | 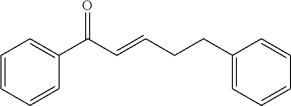 | 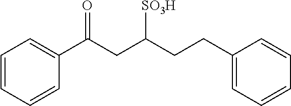 | 89 | 87 | 99.9 |
| 13 | 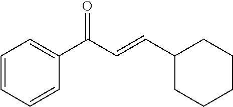 | 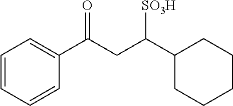 | 90 | 82 | 99.9 |
| 14 | 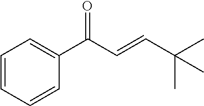 | 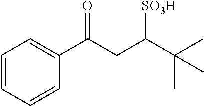 | 74 | 81 | 99.9 |
| 15 | 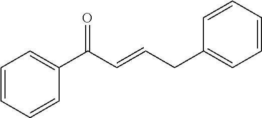 | 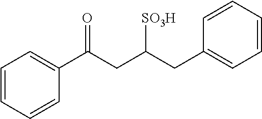 | 93 | 89 | 99.9 |
| 16 | 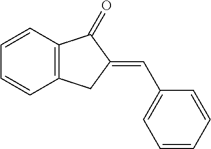 | 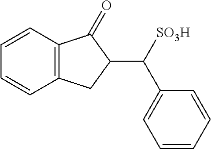 | 84 | 96 | 99.9 |
| 17 | 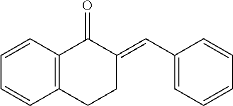 | 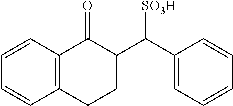 | 86 | 91 | 99.9 |
| 18 | 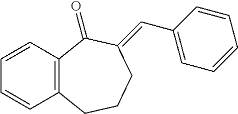 | 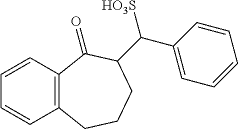 | 81 | 96 | 99.9 |
| 19 | 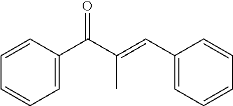 | 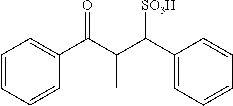 | 89 | 91 | 99.9 |
| 20 | 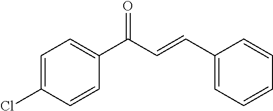 | 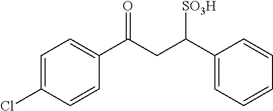 | 95 | 96 | 99.9 |
| 21 | 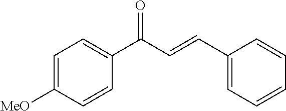 | 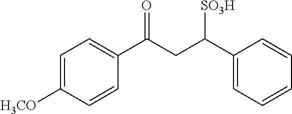 | 85 | 94 | 99.9 |
| 22 | 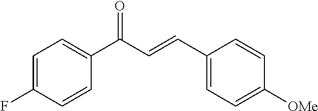 | 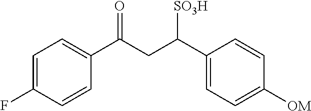 | 81 | 97 | 99.9 |
| 23 | 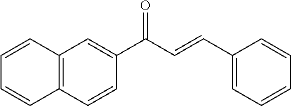 | 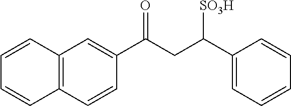 | 96 | 98 | 99.9 |
| 24 | 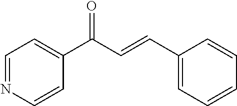 | 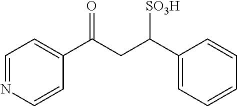 | 85 | 95 | 99.9 |
| 25 | 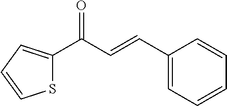 | 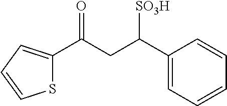 | 81 | 86 | 99.9 |
| 26 | 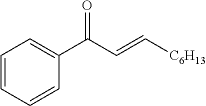 | 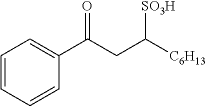 | 93 | 82 | 99.9 |

### Example 13

### Procedure for the Addition of Bisulfite to Epoxides or Aziridines

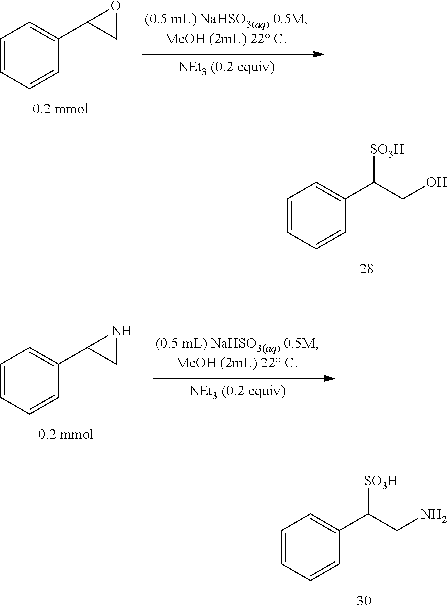

To a solution of epoxide or aziridine (0.2 mmol, 1 mmol) in MeOH (2 mL) was added aqueous NaHSO₃ (0.5M) (0.5 mL, 1.2 equiv) solution followed by triethylamine (20 mg, 0.04 mmol, 0.2 equiv) and stirred at room temperature for 16 h. After this time the reaction mixture was evaporated, the solids obtained taken up in THF/water (1/1, 3×3 mL) and the solution passed through activated DOWEX resin. The sulfonic acid was discharged from DOWEX by washing with water (3×3 mL) which was the evaporated to give colourless solid. Yield for 28 84%, yield for 30 81% yield.

1. A method for preparing sulfur-containing compounds, the method comprising reacting a donor compound comprising at least one sulfur having at least one lone pair of electrons, with an acceptor compound; wherein the reaction occurs in the presence of an amine, capable of activating the sulfur having at least one lone pair of electrons; and wherein the reaction occurs via the formation of an intermediate species, optionally a transient intermediate species, between the amine, optionally the amine catalyst, and the donor compound; and wherein the donor compound is selected from the group consisting of a sulfurous acid, a sulfenic acid and a sulfinic acid or a salt, ester or amide of the sulfurous acid, the sulfenic acid and the sulfinic acid.

2. The method according to claim 1, wherein the sulfur-containing compounds include sulfones, sulfoxides, sulfonic acids, sulfinic acids, sulfenic acids, and salts, esters thereof or amides thereof.

3. The method according to claim 1, wherein the reaction occurs in the presence of less than 0.001% (g/g) radical initiator.

4. (canceled)

5. (canceled)

6. The method according to claim 1, wherein the at least one sulfur having at least one lone pair of electrons is capable of being transferred to the acceptor compound, via the formation of an intermediate compound, or wherein the at least one sulfur having at least one lone pair of electrons is selected from a sulfite anion, a sulfonate anion or a sulfenate anion.

7. (canceled)

8. (canceled)

9. The method according to claim 1, wherein the acceptor compound comprises linear or branched unsaturated compound or a cyclic derivative thereof.

10. The method according to claim 9, wherein the unsaturated compound comprises a functional group selected from the group comprising (C═NH), (C═N—R), (C═NR₁R₂), (C═O), (C═O), (C═S), and (C═S), wherein the unsaturated compound is selected from the group comprising an imine comprising the functional group C═NH, an N-substituted imine-comprising the functional group C═N—R, an iminium ion comprising the functional group C═NR₁R₂, an aldehyde comprising the functional group C═O, a ketone comprising the functional group C═O, a thioaldehyde comprising the functional group C═S, and a thioketone comprising the functional group C═S.

11. (canceled)

12. The method according to claim 1, wherein the acceptor compound is an unsaturated compound having at least one electron-withdrawing group adjacent the at least one multiple bond wherein the at least one electron-withdrawing group is selected from carbonyl groups, and their esters; nitro groups; cyano groups; oximes; hydrazones; imino groups; protected imino groups.

13. (canceled)

14. (canceled)

15. (canceled)

16. The method according to claim 1, wherein the acceptor compound comprises an unsaturated compound selected from the group comprising, but not limited to, molecules 1a (1,3-diphenyl-propenone); 1b (3-(4-methoxy-phenyl)-1-phenyl-propenone); 1c (3-(4-nitro-phenyl)-1-phenyl-propenone); 3a (3-methyl-4-nitro-5-styryl-isoxazole); 3b (5-[2-(4-methoxy-phenyl)-vinyl]-3-methyl-4-nitro-isoxazole); 3c (3-methyl-4-nitro-5-[2-(4-nitro-phenyl)-vinyl]-isoxazole); 3d (5-[2-(4-fluoro-phenyl)-vinyl]-3-methyl-4-nitro-isoxazole); 3e (5-[2-(4-chloro-phenyl)-vinyl]-3-methyl-4-nitro-isoxazole); 5 ((2-nitro-vinyl)-benzene); 7 (but-3-en-2-one); 9 (4-methyl-pent-3-en-2-one); 11 (3-phenyl-acrylic acid ethyl ester); 13 ([1,2]naphthoquinone); 15 ([1,4]naphthoquinone); 17 (cyclohex-2-enone); 23 (3-phenyl-acrylonitrile); 48 (benzylidene-phenyl-amine), 50a (benzaldehyde), 50b (1-Phenyl-ethanone), and 52 (antraquinone sulfenic acid).

17. The method according to claim 1, wherein the catalyst is a homochiral compound or wherein the catalyst is a nucleophile comprising at least one valence electron that does not form part of a covalent bond.

18. (canceled)

19. (canceled)

20. The method according to claim 17, wherein the catalyst is a nucleophile comprising one or more hydrogen bond acceptors, and at least one hydrogen bond donor.

21. The method according to claim 17, wherein the catalyst is a nucleophile that is an amine or a salt thereof.

22. The method according to claim 21, wherein the nucleophile is a quaternary ammonium compound or a cyclic amine.

23. (canceled)

24. (canceled)

25. The method according to claim 21, wherein the amine comprises at least one nitrogen atom having at least one pair of valence electrons, wherein the at least one pair of electrons does not form part of a covalent bond; and at least three functional groups.

26. (canceled)

27. (canceled)

28. (canceled)

29. (canceled)

30. The method according to claim 1, wherein the reaction occurs in the presence of a homochiral, catalyst capable of activating the sulfur having at least one lone pair of electrons.

31. The method according to claim 30, wherein the homochiral catalyst is a homochiral amine selected from the group comprising molecules 19 (5-ethyl-1-aza-bicyclo[2.2.2]oct-2-yl)-(6-methoxy-quinolin-4-yl)-methanol); 19a (1-(3,5-bis-trifluoromethyl-phenyl)-3-[(5-ethyl-1-aza-bicyclo[2.2.2]oct-2-yl)-(6-methoxy-quinolin-4-yl)-methyl]-thiourea); 20 (pyrrolidin-2-yl-methanol); and 21 (2-amino-2-phenyl-ethanol).

32. (canceled)

33. The method according to claim 30, wherein the homochiral catalyst is a homochiral quaternary ammonium compound selected from the group comprising molecules 31 4-[(5-Ethyl-1-aza-bicyclo[2.2.2]oct-2-yl)-methoxy-methyl]-6-methoxy-quinoline, 32-[(5-Ethyl-1-aza-bicyclo[2.2.2]oct-2-yl)-methoxy-methyl]-6-methoxy-quinoline, 33 Benzoic acid (5-ethyl-1-aza-bicyclo[2.2.2]oct-2-yl)-(6-methoxy-quinolin-4-yl)-methyl ester; 34 Benzoic acid (5-ethyl-1-aza-bicyclo[2.2.2]oct-2-yl)-(6-methoxy-quinolin-4-yl)-methyl ester; 35 Benzoic acid (5-ethyl-1-aza-bicyclo[2.2.2]oct-2-yl)-(6-methoxy-quinolin-4-yl)-methyl ester; 36 Phenyl-carbamic acid (5-ethyl-1-aza-bicyclo[2.2.2]oct-2-yl)-(6-methoxy-quinolin-4-yl)-methyl ester; 37 [(5-Ethyl-1-aza-bicyclo[2.2.2]oct-2-yl)-(6-methoxy-quinolin-4-yl)-methyl]-phenyl-amine; 38 [(5-Ethyl-1-aza-bicyclo[2.2.2]oct-2-yl)-(6-methoxy-quinolin-4-yl)-methyl]-phenyl-amine; 39 1-(3,5-Bis-trifluoromethyl-phenyl)-3-[(5-ethyl-1-aza-bicyclo[2.2.2]oct-2-yl)-(6-methoxy-quinolin-4-yl)-methyl]-thiourea; 40 3-{[(5-Ethyl-1-aza-bicyclo[2.2.2]oct-2-yl)-(6-methoxy-quinolin-4-yl)-methyl]-amino}-4-phenylamino-cyclobut-3-ene-1,2-dione; 41 3-{[(5-Ethyl-1-aza-bicyclo[2.2.2]oct-2-yl)-(6-methoxy-quinolin-4-yl)-methyl]-amino}-4-phenylamino-cyclobut-3-ene-1,2-dione; 42 2-[3-(2-Amino-cyclohexyl)-thioureido]-N,N-dimethyl-propionamide; and 43 1-(3,5-Bis-trifluoromethyl-phenyl)-3-(2′-dimethylamino-[1,1′]binaphthalenyl-2-yl)-thiourea; or the group comprising, molecules 44 1-Benzyl-5-ethyl-2-[hydroxy-(6-methoxy-quinolin-4-yl)-methyl]-1-ammonium-bicyclo[2.2.2]octane chloride; 45 1-Benzyl-5-ethyl-2-(hydroxy-quinolin-4-yl-methyl)-1-ammonium-bicyclo[2.2.2]octane chloride; 46 1-Benzyl-5-ethyl-2-[hydroxy-(6-methoxy-quinolin-4-yl)-methyl]-1-ammonium-bicyclo[2.2.2]octane; and 47 1-Benzyl-5-ethyl-2-(hydroxy-quinolin-4-yl-methyl)-1-ammonium-bicyclo[2.2.2]octane chloride.

34. (canceled)

35. Sulfur-containing compounds of the formula: 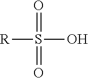
wherein R is selected from:
(a) 1-(4-Nitro-phenyl)-3-oxo-3-phenyl-propane;
(b) 2-(3-Methyl-4-nitro-isoxazol-5-yl)-1-phenyl-ethane;
(c) 1-(4-Methoxy-phenyl)-2-(3-methyl-4-nitro-isoxazol-5-yl)-ethane;
(d) 2-(3-Methyl-4-nitro-isoxazol-5-yl)-1-(4-nitro-phenyl)-ethane;
(e) 1-(4-Fluoro-phenyl)-2-(3-methyl-4-nitro-isoxazol-5-yl)-ethane;
(f) 1-(4-Chloro-phenyl)-2-(3-methyl-4-nitro-isoxazol-5-yl)-ethane; and
(g) 3-Oxo-cyclohexane.

36. Use of heterochiral, optionally homochiral, sulfur-containing compounds for the resolution of racemic mixtures of amines.

37. Use according to claim 36, wherein the homochiral sulfur-containing compounds are selected from the general formulae 4 and 2: 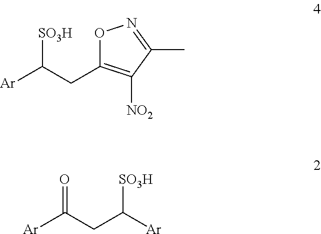
wherein Ar in either of 4 or 2 above is a saturated compound or an unsaturated compound, or a cyclic derivative thereof selected from the group comprising aromatic compounds, heteroaromatic compounds, and alkyl compounds.

38. (canceled)

39. Use according to claim 36, wherein the homochiral sulfur-containing compounds are independently selected from the group comprising molecules 4a-e, or from the group comprising molecules 2a-c, or alternatively from the group comprising molecules 51a and 51b.
